(19) United States
(12) Patent Application Publication
(10) Pub. No.: US 2018/0133162 A1
(43) Pub. Date: May 17, 2018

(54) TUNABLE POLYMERIC COMPOSITE COATING FOR CONTROLLED RELEASE

(71) Applicants: XIAO YU WU, NORTH YORK (CA); KUAN HUAN CHEN, TORONTO (CA); HAO HAN CHANG, MARKHAM (CA); ALIREZA SHALVIRI, MORRISTOWN, NJ (US); JASON SIU-WEI LI, NORTH YORK (CA)

(72) Inventors: XIAO YU WU, NORTH YORK (CA); KUAN HUAN CHEN, TORONTO (CA); HAO HAN CHANG, MARKHAM (CA); ALIREZA SHALVIRI, MORRISTOWN, NJ (US); JASON SIU-WEI LI, NORTH YORK (CA)

(21) Appl. No.: 15/810,945

(22) Filed: Nov. 13, 2017

Related U.S. Application Data
(60) Provisional application No. 62/421,082, filed on Nov. 11, 2016.

(51) Int. Cl. A61K 9/50; C09D 151/02; C08J 9/26; A61K 31/277; A61K 31/522; A61K 31/554

(52) U.S. Cl. CPC A61K 9/5036; C09D 151/02; A61K 9/5047; C08J 9/26; C08J 2201/0422; A61K 31/522; A61K 31/554; C08J 2333/12; C08J 2207/10; A61K 31/277

(57) ABSTRACT

A polymeric composite coating includes a drug release retardant polymer matrix, and pH-responsive nanoparticulate pore former. The pH-responsive pore formers function to modulate the permeability of the coating in response to pH changes which can compensate any changes in drug solubility with negligible leaching of the pore formers. The pH-responsive nanoparticulate pore formers may also function as alcohol-resistant component to the overall composite coating to resist increased solubility and permeability in presence of alcohol at 40% ethanol concentration in aqueous media. In one embodiment, the drug release retardant polymer is made of cellulose derivatives.

### FIELD

The present disclosure relates generally to the field of controlled release of ingredients in pharmaceutical formulations, nutraceuticals, animal care products, and consumer products.

### BACKGROUND

Controlled release of ingredients of interest in various products often utilizes a material such as a polymer membrane that form a barrier to transport of the ingredients from a formulation. The permeability of the membrane can be modified by adding a more permeable component to achieve desirable release kinetics. The permeability modifiers are generally called pore formers that change the porosity of the membrane. Hydrophilic, water-soluble polymers such as acrylic polymer, sodium alginate, poly(vinyl pyrrolidone) (PVP), and hydroxypropyl methyl cellulose (HPMC) have been applied as pore formers.

Although more prevalent in pharmaceuticals, uses of pore formers can also be found in food and consumer products. Pore formers have been used for sustained release of vitamins and food supplements. A specific examples include and folic acid being stored in ethyl cellulose microcapsules with sucrose as the pore former¹. Another application of pore formers can be found in bags for holding contaminated waste for steam sterilization and subsequent disposal². Carbowaxes of polyethylene glycol was used as pore formers that dissolve and form pore in the bag to allow steam to enter and sterilize the contents².

While many efforts have been made, there remains a need for formulations that can provide pH-independent drug release without complex formulation development using several different excipients and/or parameters to adjust. Conventional pore formers such as HPMC, PVP, and polyacrylic acid quickly leach out of the controlled release formulations when exposed to fluid, which would negatively impact the drug release kinetics and mechanical strength of the membrane.

A major problem encountered by the use of conventional pore former, e.g. HPMC is the high viscosity of the coating suspension, leading to process challenges. For example, addition of 10% HPMC to Surelease® (ethylcellulose) suspension increased the viscosity by over 5000 fold, which makes coating process difficult. Hence a nanoparticulate pore former with little influence on the viscosity of coating suspension is apparently advantageous.

Ionizable drugs and their salts exhibit pH dependent solubility. Hence, controlled release matrices of these drugs show pH dependent release rate in the gastrointestinal tract. In addition, gastric pH and gastric emptying are influenced by the presence of food, aging, diseases such as AIDS, and administration of acid suppressing agents such as antacids, H2-receptor antagonists (H2RAs), and proton-pump inhibitors (PPIs). A pH-dependent controlled release system could lead to inter-patient in vivo variability and bioavailability problems. Hence, greater control over release behavior of ionizable drugs and their salts assures a more reliable drug therapy³.

To overcome the problem of pH-dependent drug release two methods have been extensively studied by various research groups. One approach is based on the addition of buffering agents to the core to maintain a constant microenvironment pH and hence solubility, independent of the surrounding pH. The second approach is based on the use of permeability modifiers. The permeability modifiers are generally pore forming agents which are used to achieve a pH independent release of weakly basic drugs by leaching out and hence increasing the matrix permeability at higher pH values.

The incorporation of the pH modifiers are not as straight forward as one might initially think. The ability to improve the release properties of weakly basic and acidic drugs by adding organic buffers depends on several factors. These include the solubility of the buffers and the buffering capacity together with the pKₐ values and the solubility of the salts formed with the drug. The solubility of the buffers plays an important role in maintaining a target pH value inside the dosage form. For example acids without adequate solubility, fumaric acid, only exert an effect for a limited duration⁴. Highly soluble acids such as tartaric and citric acids diffuse too rapidly with the drug through the film-coating. Often higher amounts of acids (up to 500% relative to the drug) have to be used to achieve a pH-independent drug release over a long period of time⁵. Akiyama et al showed that incorporation of highly soluble pH modifiers are ineffective in controlling the pH microenvironment of microspheres of a weakly basic drug as these excipients diffused out of the matrix at a faster rate compared to the drug itself. An insoluble pH modifier such as magnesium oxide proved to be effective in achieving a pH-independent release profile as this excipient would stay in the matrix long enough to maintain its effect on pH. However, it has to be noted that a magnesium oxide to drug ratio of at least 3 to 1 is required to achieve a pH-independent release⁶.

A pH-independent controlled release formulations of divalproex sodium, a weakly acidic drug, were developed using Eudragit® E100 or Fujicalin® as release modifiers. The formulations with Eudragit® E100 showed pH independent release mainly due to increase in the pH microenvironment of the swollen gel layer. Fujicalin was less effective in achieving a pH independent release. This was attributed to the relative inability to elevate the pH and shorter residence time of Fujicalin in the matrix relative to Eudragit® E100⁷. Aditya et al. investigated the effect of incorporating a polymeric pH modifier vs. an organic acid on the release behavior of trimethoprim (pKₐ=6.6) matrix tablets⁸. Incorporation of acrylic acid methacrylate co-polymer (Eudragit® L100-55) had marginal effect on release behavior as the pH modulation effects were neutralized by the basicity of the drug. In addition, water uptake and scanning electron microscopy (SEM) studies suggested that Eudragit® L100-55 incorporation also resulted in reduced water uptake and matrix permeability. On the other hand, the reduction in the microenvironment pH brought on by the incorporation of melanic acid was sufficiently high and persistence to result in pH-independent release⁷.

Another approach to develop pH-independent controlled release systems for weakly basic drugs is to incorporate ingredients which increase the porosity of the system, mostly by leaching out, to compensate for the reduction in the solubility. Incorporation of poly(methacrylic acid) into the papaverine microspheres, a basic drug, resulted in pH-independent release. SEM investigations revealed that the acrylic polymer acted as a pore former at higher pH's and hence corrected for the reduction in drug solubility by increasing the permeability of the matrix leading to a constant release rate. Kohri et al. developed pH-independent sustained release granules of dipyridamole which is a weakly basic drug⁹. The granules contained drug, carboxymethyl ethyl cellulose (CMEC), hydroxypropyl methyl cellulose (HPMC), and Eudragit® RS100, and exhibited pH independent release in pH 1.2-7. The authors proposed that the drug release from such matrices below pH 4 should be improved by dissolving HPMC and suppressed by CMEC and Eudragit® RS 100, while that above pH 5 should be enhanced by dissolving HPMC and CMEC and suppressed by Eudragit RS 100. Comparison of this formulation with a commercial pH-dependent controlled release formulation in controlled gastric acidity rabbits revealed that the pH-independent granules produce drug plasma level concentrations with less degree of variability compared to the conventional formulation⁹. A controlled release matrix composed of a weakly basic drug, a hydrophilic polymer (HPMC E-50) and an enteric polymer (Eudragit® L-100-SS) was developed by Oren and Seidler¹⁰. The enteric polymer is insoluble at low pH and acts as a part of the matrix retarding the drug release. At higher pH Eudragit® L-100-SS dissolves which increases the permeability of the dosage form to correct for reduction in drug solubility, and hence a pH-independent release system is achieved¹⁰. A pH-independent controlled release system was developed by Howard and Timmins which was composed of sodium alginate, an anionic polymer, and HPMC¹¹. At low pH sodium alginate precipitates in the gel layer providing resistance of this layer to erosion. At higher pH values, the alginate forms a soluble salt and hence undergoes erosion. As a result, with increase in pH the release mechanism changes from predominately diffusion controlled to erosion controlled, and the resulting higher permeability of the gel layer makes up for the reduction in the solubility¹¹. A multiparticular device coated with walls of controlled porosity was introduced as an approach to delivery of weakly basic compounds. When exposed to water, low levels of a soluble additive were leached from semi-permeable polymeric coat leading to increased porosity. The sponge like structure forms a controlled porosity wall, permeable to both water and the dissolved drug, thereby providing pH-dependent drug release¹².

Existing polymers with pH-dependent solubility, such as methacrylic acid-ethyl acrylate copolymer (Eudragit® L) and hypromellose acetate succinate, are typically used as pore former materials for more specific pH-sensitive applications, e.g. protection of acid-labile drugs, delayed release, targeted delivery to given regions of the gastrointestinal (GI) tract¹³⁻²³. These polymers are water-insoluble at low pH in the stomach and remain within the coating thus hindering drug release. At higher pH in the small and large intestines, they become water-soluble and leach out from the coating, resulting in a more porous and permeable film^{16,24-27}. This pH-dependent leaching could reduce the mechanical strength of the film and compromise coating integrity, which can increase the risks of premature drug release and dose dumping in the GI tract, leading to unwanted toxicity^{27,28}. Furthermore, the films could lose the ability to continuously modulate drug permeability once the pore former has leached out. Another problem with use of water-soluble polymers as pore former is the significantly increased viscosity of the coating dispersion. High viscosity of the coating dispersion can cause clogging of equipment parts (i.e. spray nozzle and tubing) and inconsistency in the coating^{29,30}.

Alcohol-induced dose dumping (ADD) of modified release (MR) dosage forms is of particular high risk for opiate drugs and drugs with narrow therapeutic windows. As MR dosage forms contain a large dose, a sudden release, known as dose dumping, can happen when alcohol is co-ingested if the dosage form is not resistant to alcohol. Loss of delayed release and prematured drug release than intended can lead to risks of toxicity or side effects. Opiate drugs, when taken in significantly high amounts, can result in side effects, such as severe itching, vomiting, nausea, urinary retention, and respiratory depression³¹. Alcohol resistance of dosage forms is also important for drugs with narrow therapeutic index, such as theophylline³². Such drugs can produce severe side effects if dose dumping occurs, and hence should be carefully noted. In 2005, the ongoing concern has led the U.S. Food and Drug Administration (FDA) to withdraw Palladone™, a controlled release formulation of hydromorphone, from the market due to increase in fatal risks³³. In addition, alcohol has shown to prolong or accelerate gastric emptying rate depending on the alcohol concentration, hence an alcohol resistant and pH-responsiveness technology is necessary³⁴⁻³⁶.

Various approaches have been attempted to reduce ADD. One approach is the utilization of alcohol insoluble components within the matrix or core composition. The added component is resistant to alcohol due to their lack of solubility and ability to alter swellability, thereby, limiting influx of hydroalcoholic media. Another approach is based on the addition of alcohol-insoluble component to conventional film coating, such as ethylcellulose. As the conventional coating is soluble in ethanol but insoluble in aqueous media. The added alcohol-insoluble component will act to compensate the ethanol solubility of the traditional film coating^{37,38}.

However, matrix system may not be preferred at times compared to coating system. For example, controlled release or, in this particular instance, the alcohol resistance of a matrix or core composition system may depend on several factors involved, including compressibility, hardness, disintegration time, excipient compatibility, etc. In addition, a coating system can achieve zero order release, whereas matrix systems cannot.

One coating technology for preventing ADD is Aquacoat® ARC. Aquacoat® ARC has been developed by blending guar gum (soluble in water, insoluble in ethanol) and ethyl cellulose (insoluble in water, soluble in ethanol) as alcohol-resistant film coating³⁹⁻⁴². Aquacoat®ECD30 acts as component 1 while guar gum acts as component 2 to compensate the solubility of the other within the hydroalcoholic media. An investigation into the formulation parameters reported that the incorporation of at least 5% guar gum and apparent viscosity of great than 150 cP (of 1% aqueous guar gum) is needed to gain alcohol-resistant properties³⁹. A major problem encountered by the use of guar gum is the high viscosity of the coating suspension, leading to process challenges. For example, addition of 12% guar gum to Surelease® (ethylcellulose) suspension increased the viscosity drastically, which makes coating process difficult. Uneven and non-homogeneous coating of dosage form can increase the risk of alcohol dose dumping. Another issue with guar gum is its aqueous solubility. As guar gum is very soluble in water, the loss of guar gum in aqueous media is quite significant, which can negatively impact the rate of drug release, synonymous to the leaching of water-soluble pore formers. Therefore, a nanoparticulate pore former with little impact on the viscosity of coating suspension and negligible leaching is more advantageous.

### SUMMARY

A polymeric composite coating for pH-independent controlled release of weakly basic and acidic drugs has now been developed that functions to modulate the permeability of the coating continuously and automatically in response to the varying pH of the gastrointestinal tract.

In one aspect of the disclosure, a polymeric composite coating is provided comprising a drug release retardant polymeric matrix and pH-responsive nanoparticulate pore formers that function to modulate the permeability of the overall composite coating in response to changes in pH throughout the gastrointestinal tract with negligible leaching of the nanoparticulate pore formers.

According to one embodiment, the drug release retardant polymer matrix comprises any one or a combination of cellulose derivatives, (alkyl) acrylate polymers and derivatives, polyvinyls and copolymers.

In one embodiment, the pH-responsive nanoparticulate pore former comprises a first polymer grafted to a second polymer, which is covalently bound to a third polymer.

In one embodiment, the pH-responsive nanoparticulate pore former comprises of a biocompatible polysaccharide (such as starch) backbone grafted with a stabilizing emulsifier and a pH-responsive polymer adapted to alter the porosity of the polymeric coating, in which the nanoparticulate pore former is incorporated into, in response to pH changes in gastrointestinal tract.

According to one embodiment of the disclosure, the first polymer comprises a polysaccharide; the second polymer is a crosslinked polymer comprising of a ionizable polymer; and the third polymer is a polysorbate comprising a (C9-C31)R—C(O)O— group covalently bound to the second polymer by a C—C bond between the carbon backbone of the second polymer and the R group. According to one embodiment, the ionizable polymer is any one of polymethacrylic acid, polyacrylic acid, and maleic acid copolymers, and polyvinyl derivatives. According to a further embodiment, the ionizable polymer is selected from methacrylic acid-ethacrylate copolymer, poly(2-(dimethylamino)ethyl methacrylate), poly(2-(diisopropylamino)ethyl methacrylate), poly(2-n-morpholinoethyl methacrylate), poly(l-vinylimidazole), poly(2-vinylpyridine), and (4-vinylpyridine).

In a further aspect, a method of achieving pH-independent release of a weakly basic or acidic drug has been provided comprising of applying a polymeric composite coating onto drug-loaded beads, wherein the coating comprises a drug release retardant polymer matrixand pH-responsive nanoparticulate pore formers that functions to modulate the permeability of the coating in response to low and high gastrointestinal pH in order to compensate any changes in drug solubility.

In yet another aspect, a method of achieving alcohol-resistant release of drugs has been provided, which comprises of application of an alcohol-resistant polymeric coating, wherein a nanoparticulate pore former functions to resist ADD throughout the gastrointestinal tract with negligible leaching of the nanoparticulate pore former in the presence of alcohol.

These and other aspects of the present disclosure will become apparent from the following detailed description by reference to the figures. It should be understood, however, that the detailed description and the specific examples while indicating preferred embodiments of the disclosure are given by way of illustration only, since various modifications and changes within the spirit and scope of the disclosure will become apparent to those skilled in the art from this detailed description.

### BRIEF DESCRIPTION OF THE FIGURES

FIG. 1 graphically illustrates the relative viscosities of 15% w/v Surelease® dispersion without pore formers as control and 15% w/v Surelease® dispersion with either the terpolymer, PVP, Eudragit L, or HPMC (n=3);

FIG. 2 graphically illustrates the tensile strength of wet control membrane at 0% pore former level and wet composite membrane at 10% pore former level that were immersed in pH 6.8 phosphate buffer over 24 hours (n=3);

FIG. 3 graphically illustrates the Young's modulus of wet control membrane at 0% pore former level and wet composite membrane at 10% pore former level that were immersed in pH 6.8 phosphate buffer over 24 hours (n=3);

FIG. 4 shows representative SEM photographs of the cross-sectional area of the control membrane and the composite membranes at 5%, 10%, and 15% pore former levels of terpolymer;

FIG. 5 shows representative SEM photographs of the surfaces of diltiazem HCl beads coated with the composite membrane at 5%, 10%, and 15% pore former levels and beads coated with Surelease and HPMC at 15% pore former level. The inserts are close-ups of the surface at 850× magnification;

FIG. 6 graphically illustrates the glass transition temperature (T_{g}) of the control membrane, the composite membranes at 5%, 10%, and 15% pore former levels, and pure terpolymer nanoparticles (n=3);

FIG. 7 graphically illustrates the permeability of verapamil HCl at pH 1.2 and pH 6.8 of control membrane and composite membranes at 5% and 10% pore former level (n=3);

FIG. 8 graphically illustrates the permeability of theophylline at pH 1.2 and pH 6.8 of control membrane and composite membrane at 10% pore former level (n=3);

FIG. 9 graphically illustrates the swelling ratios of control membrane and composite membrane at 10% pore former level at pH 1.2 and 6.8 (n=3);

FIG. 10 graphically illustrates fractional release over time of a weakly basic drug, diltiazem HCl, in 0.1 N HCl and pH 6.8 phosphate buffer from drug-loaded beads coated with the composite coating with 5% pore former level of the terpolymer (n=3);

FIG. 11 graphically illustrates fractional release over time of a weakly basic drug, diltiazem HCl, in 0.1 N HCl and pH 6.8 phosphate buffer from drug-loaded beads coated with the composite coating with 10% pore former level of the terpolymer (n=3);

FIG. 12 graphically illustrates fractional release over time of a weakly basic drug, diltiazem HCl, in 0.1 N HCl and pH 6.8 phosphate buffer from drug-loaded beads coated with the composite coating with 15% pore former level of the terpolymer (n=3);

FIG. 13 graphically illustrates fractional release over time of a weakly basic drug, diltiazem HCl, in 0.1 N HCl and pH 6.8 phosphate buffer from drug-loaded beads coated with the composite coating with 15% pore former level of HPMC (n=3);

FIG. 14 graphically illustrates the change in diameter of PDEAEM-g-starch nanoparticles as a function of pH of the medium (n=3);

FIG. 15 shows representative SEM photograph of the cross-sectional area of the PDEAEM-g-starch nanoparticle-embedded ethylcellulose composite membrane;

FIG. 16 shows representative SEM photograph of the PDEAEM-g-starch nanoparticles embedded within the ethylcellulose membrane;

FIG. 17 shows kinetics of theophylline release across PDEAEM-g-starch nanoparticles embedded ethylcellulose membrane (N=3)

FIG. 28 shows kinetics of verapamil HCl release across PDEAEM-g-starch nanoparticles embedded ethylcellulose membrane (N=3)

FIG. 39 shows kinetics of ibuprofen release across PDEAEM-g-starch nanoparticles embedded ethylcellulose membrane (N=3)

FIG. 20 shows kinetics of vitamin B12 release across PDEAEM-g-starch nanoparticles embedded ethylcellulose membrane (N=3)

FIG. 21 graphically illustrates the permeability of diltiazem HCl at pH 1.2 and pH 6.8 of Eudragit L membrane and composite membranes (TPN) at 10% pore former level (n=3)

FIG. 22 graphically illustrates the weight loss of blank (open circles), 10% TPN (grey squares), or 10% Eudragit® L (filled triangles) films immersed in pH 1.2 HCl solution over 24 hours (n=3)

FIG. 23 graphically illustrates the weight loss of blank (open circles), 10% TPN (grey squares), or 10% Eudragit® L (filled triangles) films immersed in pH 6.8 phosphate solution over 24 hours (n=3)

FIG. 24 shows representative SEM photographs of blank, 10% TPN, and 10% Eudragit® L films after being immersed in pH 1.2 HCl solution and pH 6.8 phosphate buffer solution over time.

FIG. 25 graphically illustrates the permeability of theophylline at 0% and 40% ethanol concentration in 0.1 N HCl solution through blank Surelease membrane and terpolymer membrane at 10% pore former level (n=3)

FIG. 26 graphically illustrates the weight loss of blank, 10% TPN, or 12% guar gum films at 0% and 40% ethanol concentration in 0.1 N HCl solution over 4 hours (n=3)

FIG. 27 graphically illustrates the medium uptake of blank membrane, 10% TPN, and 12% guar gum films at 0% and 40% ethanol concentration in 0.1 N HCl solution over 4 hours (n=3)

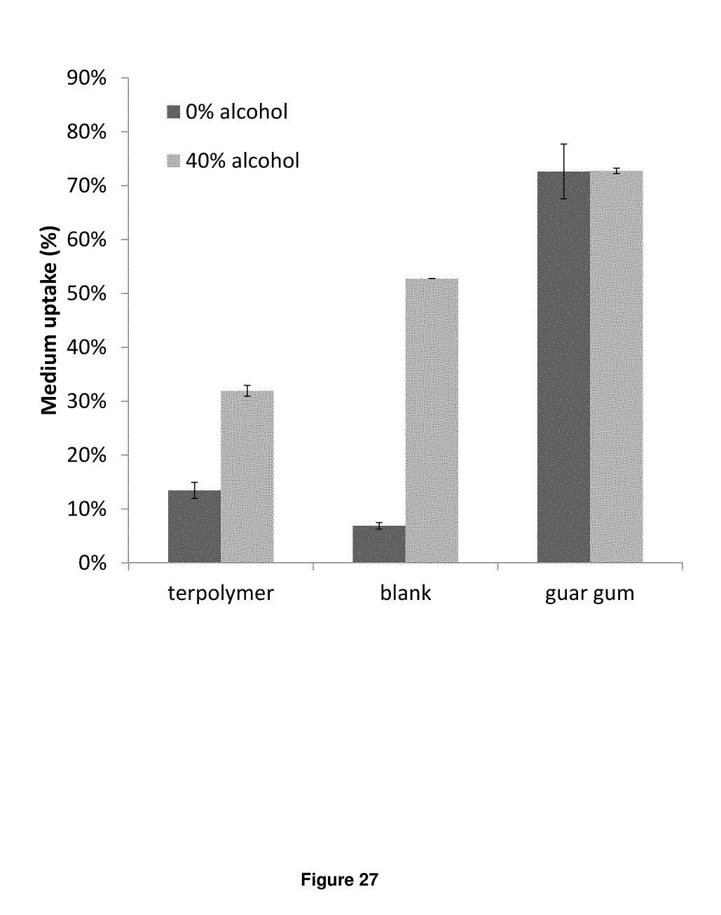

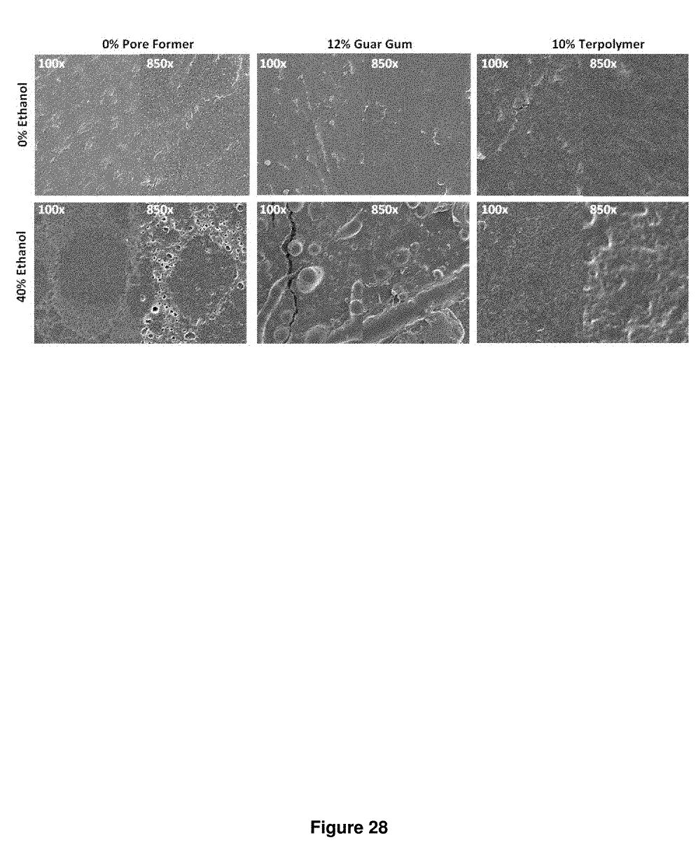

FIG. 28 shows representative SEM photographs of blank, 10% TPN, and 12% guar gum films after being immersed in 0% and 40% ethanol concentration in 0.1 N HCl (pH 1.2) solution over 4 hours at 100× and 850× resolution

### DETAILED DESCRIPTION OF THE DISCLOSURE

A polymeric composite coating for pH-independent controlled release of weakly basic and acidic drugs is provided comprising of release retardant polymeric coating, wherein pH-responsive nanoparticulate pore formers functions to modulate the permeability of the coating in response to low and high gastrointestinal pH in order to compensate any changes in drug solubility.

The term “drug release retardant polymer” as used herein means any polymer that retards the release of a drug (or any active ingredients in pharmaceutical formulations, nutraceuticals, animal care products, and consumer products).

A pH-sensitive polymeric nanoparticulate system is provided composing of three polymeric components: an ionizable polymer (for example, PMAA), polysorbate 80 (PS 80) and starch. The terpolymeric system (terpolymer) can be incorporated as a pH-responsive pore former into existing commercial controlled release polymers such as Surelease®. PMAA, PS80, and starch are all generally regarded as safe by the FDA. Due to its ease of chemical modification and high biocompatibility, starch is used as the backbone of the terpolymer for which the PMAA and PS80 are grafted on. PMAA is the pH-sensitive component of the nanoparticles, while PS 80 helps stabilize the nanoparticles. PS80 is also a non-ionic surfactant used as a solubilizing agent and permeation enhancer in pharmaceutical preparations.

The polymeric composite coating can be applied onto tablets and beads loaded with weakly basic or acidic drugs with pH-dependent solubility in order to achieve pH-independent controlled release of these drugs. The terpolymer nanoparticles incorporated within the composite coating function as pH-responsive pore formers by modulating the coating porosity in response to low and high gastrointestinal pH in order to compensate any changes in drug solubility due to changes in pH. The extremely small size of the terpolymer nanoparticles makes them ideal as pH-responsive pore formers due to the highly increased surface area that enables fast responsiveness to changes in gastrointestinal pH.

The ability of the terpolymer to modulate permeability in response to varying pH is due to the ionizable polymer component of the terpolymer, which unionizes at low pH and ionizes at high pH. In an embodiment of the present disclosure, the ionizable polymer may be polymethacrylic acid derivatives, acrylic acid derivatives, maleic acid copolymers, or polyvinyl derivatives. For example, the ionizable polymer may be any one of poly(methacrylic acid), poly(acrylic acid), methacrylic acid-methacrylate copolymer, methacrylic acid-ethacrylate copolymer, poly(2-(dimethylamino)ethyl methacrylate), poly(2-(diisopropylamino)ethyl methacrylate), poly(2-n-morpholinoethyl methacrylate), poly(1-vinylimidazole), poly(2-vinylpyridine), and (4-vinylpyridine). According to one embodiment, the ionizable polymer is PMAA, i.e., poly(methacrylic acid). The degree of ionization of the PMAA changes the porosity of the composite coating by either dehydrating the composite coating at low pH or hydrating the composite coating at high pH. Hydration of the composite coating increases the motility and free volume of the polymeric chains as well as induces the formation of water channels, where drug diffusivity is much higher than in the polymer network.

Broadly stated, the present disclosure relates to a polymeric composite coating with a pH-responsive pore former component. The polymeric composite coating is ideal for pharmaceutical formulations that require pH-independent release of weakly basic or acidic drugs.

Embodiments of the disclosure are described by reference to the following specific examples which are not to be construed as limiting.

### Example 1—Synthesis of Terpolymer Nanoparticles

An aqueous based free radical dispersion polymerization process using potassium persulfate (KPS)/sodium thiosulfate initiator system (STS) was used to prepare the PMAA-PS80-g-starch nanoparticles in an one-pot synthesis. The polymerizations were conducted in a 250 mL two-necked flask immersed in a water bath with nitrogen inlet, a condenser, and magnetic stirrer. The molar ratio of MAA:N,N′-methylenebisacrylamide (MBA):PS80:starch used as the feed composition for nanoparticle synthesis was 1.00:0.139:0.0248:0.0212. 4.9 mmol of soluble starch was first dissolved in 180 mL of distilled deionized water (DDIW) at 90° C. for 30 minutes. The solution was then cooled down to 65° C. and purged with nitrogen for 30 min to remove any dissolved oxygen. After purging the starch solution, 0.45 mmol of KPS and 1.36 mmol of STS dissolved in 5 mL of DDIW were added to the flask and stirred for 10 minutes. Next 0.69 mmol of SDS dissolved together with 0.57 mmol of PS80 in 10 mL of DDIW were added. Finally, 23.1 mmol of MAA and 3.2 mmol of MBA dissolved in 10 mL of DDIW were added to the flask to start the reaction. The reaction was carried out for 12 hours at 65° C. to ensure complete conversion. Following the synthesis, the product was neutralized with 1 N NaOH and ultra-centrifuged (Beckman Coulter, CA, USA) at 35,000 rpm for 40 minutes and freeze-dried for storage.

### Example 2—Preparation of Free Films of Polymeric Composite Coating

Free films of polymeric composite coating were prepared by casting Surelease® ethylcellulose dispersion (grade E-7-19040) mixed with the nanoparticles. Nanoparticles at 10% w/w based on the dry ethylcellulose weight were dispersed in 10 mL DDIW. The mixture was stirred overnight and dispersed with the Ultrasonic Processor (UP100H, Hielscher, Teltow, Germany) for 15 minutes. Surelease® was then added to the mixture at 15% w/v and stirred for 2 hours. The mixture was then poured onto a polytetrafluoroethylene evaporating plate and degassed under vacuum for 30 minutes. After degassing, the evaporating plate was dried at 37° C. for 48 hours. Once dried, the membrane was removed from the evaporating plate and stored at room temperature. Surelease® membranes with HPMC were prepared in the same manner.

### Example 3—Determination of Viscosity of Polymeric Composite Coating Dispersion

Viscosity of polymeric composite coating dispersion was determined in order to assess its ease of use during the coating process. 5%, 10%, and 15% w/w of terpolymer based on dry ethylcellulose weight in 15% w/v Surelease® dispersions were compared to 15% w/w HPMC, 15% w/w PVP, and 15% w/w Eudragit L in 15% w/v Surelease® dispersions and also to 15% Surelease® dispersion without pore formers as control. The relative viscosities (ηrel) were measured with a capillary viscometer after calibration with DDIW.

### Example 4—Determination of Mechanical Properties of Polymeric Composite Coating

Mechanical properties such as tensile strength and Young's modulus of dry and wet composite membranes were determined by using a universal testing system Instron 3366 with a 10 kN capacity load cell and a cross-head of 0.05 mm/s. Dry and wet membrane sample were cut with a ASTM D-638 Type V specimen cutting die. Dry and wet samples were then secured by rubberized turn-screw vise grips and properly aligned before the start of the test. Dry membranes at 5% and 10% pore former levels of terpolymer nanoparticles were stored at 21° C. and 45% RH for 24 hours prior to testing to equilibrate the specimen to testing conditions. Wet samples at 10% pore former level were immersed in phosphate buffer at 37° C. Specimens were cut and tested after 4, 8, and 24 hours of soaking to evaluate the effect of aqueous medium on mechanical properties of the composite membrane over time.

### Example 5—Determination of Glass Transition Temperature of Polymeric Composite Coating

The glass transition temperatures (T_{g}) of composite membranes at 5%, 10%, and 15% pore former levels were determined using differential scanning calorimetry (TA Instruments 2010 DSC, USA). Approximately 7 mg of sample were sealed in standard aluminum pans and heated from 30° C. to 120° C. at a heating rate of 10° C./min in an atmosphere of nitrogen.

### Example 6—Determination of pH-Sensitive Permeability of Polymeric Composite Coating

The pH-dependence of the permeability of terpolymer composite membranes at 5% and 10% pore former level was determined using standard 3.4 mL Side-Bi-Side diffusion cells (PermeGear, Hellertown, Pa., USA). The membranes were pre-swollen in either pH 1.2 HCl solution or pH 6.8 phosphate buffer and the thickness of the pre-swollen disks were measured using a micrometer. The pre-swollen disks were inserted between well-stirred diffusion cells kept at 37° C. The receptor cell contained either HCl solution or phosphate buffer, while the donor cell contained 1 mg/mL of either verapamil HCl, theophylline, or diltiazem HCl dissolved in either pH 1.2 HCl solution or pH 6.8 phosphate buffer. Drug concentration in the receptor cell were measured using a UV-Vis spectrophotometer (8453, Agilent, Waldbronn, Germany).

### Example 7—Determination of Swelling Properties of Polymeric Composite Coating

The change in weight of the membrane samples due to the uptake of water were measured over time. Samples were cut from composite membranes of 10% pore former level and placed in either pH 1.2 HCl solution or pH 6.8 phosphate buffer at 37° C.

### Example 8—Drug Layering of Diltiazem HCl onto Microcrystalline Cellulose Beads

The composition of the drug solution is listed in Table 1. The drug solution was prepared by mixing diltiazem HCl with PVP in DDIW. Drug layering of the microcrystalline cellulose (MCC) beads was performed using a fluid bed dryer assembled with a bottom spray Wurster apparatus (Pro-C-ept Formate 4M8 Fluid Bed, Zelgate, Belgium) and a nozzle size of 0.8 mm. Coating parameters used were: inlet temperature of 50° C.; air speed of 1.0 m³/min; air nozzle pressure of 0.25 bar; and spray rate of 1 g/min.

TABLE 1
Composition of diltiazem HCl drug solution.

| Materials | % w/w | Wt (g)/220 g batch | Dry wt (g) |
|---|---|---|---|
| PVP | 2 | 4.4 | 4.4 |
| Diltiazem HCl | 10 | 22 | 22 |
| DDIW | 85 | 193.6 | N/A |

### Example 9—Application of Polymeric Composite Coating onto Drug-Layered Beads

The compositions of the polymeric composite coating dispersions are listed in Table 2. 5% solutions of terpolymer were prepared by mixing the pore formers in water for 12 hours. The 5% terpolymer solution was further dispersed with the Ultrasonic Processor (UP100H, Hielscher, Teltow, Germany) for another 30 minutes. the polymeric composite coating dispersions were prepared by adding the 5% solution of either pore formers to Surelease® ethylcellulose dispersion (grade E-7-19040) until the target pore former level (5, 10, or 15% based on dry ethylcellulose weight) and then adding enough DDIW water to dilute the ethylcellulose content to 10%. Coating of drug-layered MCC beads was performed using the using a fluid bed dryer assembled with a bottom spray Wurster apparatus (Pro-C-ept Formate 4M8 Fluid Bed, Zelgate, Belgium) and a nozzle size of 0.8 mm. Coating parameters used were: inlet temperature of 30° C.; air speed of 0.35 m³/min; air nozzle pressure of 0.375 bar; and spray rate of 1 g/min. 90 g batches of drug-layered beads were coated to 20% weight gain. After coating, the finished beads were cured for 24 hours at 60° C.

TABLE 2
Composition of dispersion of polymeric composite coating.

| Materials | % w/w | Wt (g)/~227 g | Dry wt (g) |
|---|---|---|---|
| Surelease ® | 40 | 90 | 22.5 |
| Terpolymer | 0.5/1/1.5 | 1.125/2.25/3.375 | 1.125/2.25/3.375 |
| DDIW | 60 | 135 | N/A |

### Example 10—Dissolution Study of Coated Drug Beads

Release of diltiazem HCl from the coated beads was determined using an USP dissolution apparatus I (VanKel VK7000, Varian Inc., Edison, N.J., USA) and an UV-Vis spectrophotometer (8453, Agilent, Waldbronn, Germany). 0.5 g of coated beads were placed in baskets and immersed in 900 mL of 0.1 N HCl or pH 6.8 phosphate buffer at 37° C. and rotated at 100 rpm.

### Example 11—Synthesis of PDEAEM-g-Starch Nanoparticles

Briefly, 4 g of maltodextrin was added to 240 mL of water in a round-bottom, two-mouthed flask. The solution was placed in a water bath, stirred and placed under an N₂ purge until the temperature of the mixture had reached no less than 60° C., up to a final temperature of 70° C. Upon reaching 60° C., 0.4 g of 2,2′-azobiz(2-methylpropioniamidine) dihydrochloride was added, followed by 0.4 g of PVP in 10 mL of water. 4 g of 2-(diethylamine) ethylmethacrylate (DEAEM), and 100 μL of ethylene glycol methacrylate (EGDM) in 10 mL ethanol were then added to the mixture to initiate polymerization, and the flask was sealed and connected to a water condenser. The mixture was left at 70° C. for 8 hours in an N₂ blanket, and left to stir overnight. Once polymerization reached completion, the dispersion was dialyzed in filtered water in 12,000-14,000 MWCO Spectra/Por® dialysis tubing for 24 hours. After dialyzing, the mixture was centrifuged at 45,000 RPM at 3° C. for 30 minutes to obtain a pellet. The pellets were then lyophilized and stored for future use.

### Example 12—Characterization of PDEAEM-g-Starch Nanoparticles Using Dynamic Light Scattering

Lyophilized nanoparticles were reconstituted in phosphate buffer to create a 1 mg/mL solution. Nanoparticle solutions were further diluted in pH 5.5, 6.0, 6.5 and 7.4 phosphate buffers to test pH-sensitivity. Particle size was determined using a Zeta Potential/Particle Sizer NICOMP 380 ZLS (PSS/NICOMP Particle Sizing Systems, Santa Barbara, Calif.). Intensity of the laser was maintained at or below 200 mHz during measurements.

### Example 13—Preparation of PDEAEM-g-Starch Nanoparticle-Embedded Ethylcellulose Composite Membrane

0.270 g of Ethocel (75 cP) and 0.198 g of dried nanoparticles were added to 8.50 mL ethanol and stirred until homogenous. 0.059 mL of dibutyl sebacate was added to the mixture and left to stir overnight to partition into polymer phase. In the subsequent day, the mixture was cast in a 12 mm-diametre Teflon dish and placed in a desiccator to cast overnight at 23° C.

### Example 14—Determination of Leaching of Pore Formers from Polymeric Composite Coating

Changes in the dried weights of the membrane samples due to pore former leaching were measured. Dry samples from the blank, 10% TPN, and 10% Eudragit® L films were weighed and then placed in either pH 1.2 HCl solution or pH 6.8 phosphate buffer at 37° C. under constant shaking. At predetermined time points, the samples were removed and dried at 50° C. for 24 hours and weighed to get weight loss. The morphological structures of blank, 10% TPN, and 10% Eudragit® L films were examined by SEM in their initial dry state or after immersion in pH 1.2 HCl solution or pH 6.8 phosphate buffer. Samples were thoroughly dried, freeze-fractured, and gold coated before mounted onto sample holders with double-sided tapes. The SEM photographs were obtained using a Hitachi-3400 microscope at 5 kV.

### Example 15—Determination of Alcohol Resistance of Polymeric Composite Coating

Alcohol resistance of the coating was evaluated by weight loss and drug permeability tests before and after immersed in ethanol aqueous solutions. Changes in the dried weights of the membrane samples due to pore former leaching were measured. Dry samples from the blank, 10% TPN, and 12% guar gum films were weighed and then placed in either 0% or 40% ethanol concentration 0.1 N HCl at 37° C. under constant shaking. At 4 hours, the samples were removed and dried at 50° C. for 24 hours and weighed to get weight loss. The morphological structures of blank, 10% TPN, and 12% guar gum films were examined by SEM in their initial dry state or after immersion in either 0% or 40% ethanol concentration 0.1 N HCl. Samples were thoroughly dried and gold coated before mounted onto sample holders with double-sided tapes. The SEM photographs were obtained using a Hitachi-3400 microscope at 5 kV. Permeabilities of the blank and composite membranes were determined at in both 0% or 40% ethanol concentration 0.1 N HCl. Theophylline, a neutral drug, was used as a model drug.

### Results

Viscosity of Polymeric Composite Coating Dispersion:

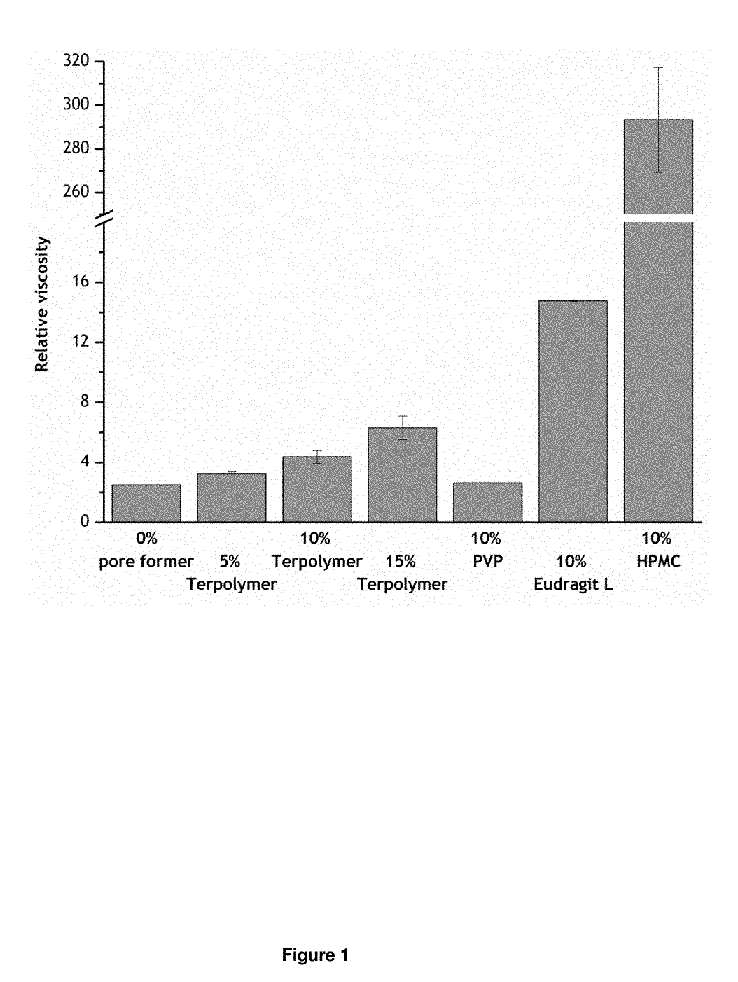

The viscosity of the polymeric composite coating dispersion was determined in order to assess the ease of use during the coating process. Coating dispersions with high viscosity can clog equipment parts such as the spray nozzle and tubing and can also negatively affect the uniformity of the coating on individual beads as they cannot evenly spread on the bead surface. Coating dispersions with HPMC have very high viscosities due to the high solubility of HPMC in water even at low concentrations. As shown in FIG. 1, Eudragit L and HPMC at 10% pore former level increased the viscosity of 15% w/v Surelease dispersion by 490% and 5560%, respectively. On the other hand, terpolymer nanoparticles and PVP at the same pore former level only increased the viscosity by 75.5% and 5.35%, respectively. As pore former levels of the terpolymer nanoparticles increased from 0% to 15%, the viscosity increased in an approximately linear fashion. HPMC polymer chains are relatively straight and uncoiled when dissolved in water which greatly increases the viscosity of the solution, whereas the terpolymer chains are cross-linked to form more compacted nanoparticles that do not heavily impact the viscosity of the solution.

Mechanical Properties of Polymeric Composite Membrane:

The mechanical properties of the composite membranes at 5% and 10% pore former levels were evaluated, whereas composite membrane at 15% pore former level and membrane with HPMC could not be tested due to cracks and defects of their free films. Table 3 shows the tensile strength and Young's modulus of the control and membrane composite membranes, which were calculated from the applied load versus extension profile.

TABLE 3
Comparisons of mechanical properties between control
membrane with no pore former and composite membranes
at 5% and 10% pore former levels (n = 3).

| Membrane | Tensile Stress (MPa) | Young's Modulus (MPa) |
|---|---|---|
| Control | 4.3 ± 0.3 | 56.9 ± 0.9 |
| 5% terpolymer | 3.6 ± 0.3 | 57.5 ± 3.2 |
| 10% terpolymer | 3.8 ± 0.5 | 71.0 ± 4.7 |

A soft and weak polymer is characterized by low values in tensile strength, elongation at break, and Young's modulus, while a hard and strong polymer is characterized by high values in these properties. A polymer that is both soft and strong is characterized by low Young's modulus, moderate tensile strength, and high elongation at break. The tensile strengths of the dry control dry and the composite membranes at 5% and 10% pore former levels were very similar. The difference in tensile strength between the composite membrane and the control membrane were statistically insignificant. The Young's modulus was found to be statistically significantly higher for the dry composite membrane at 10% pore former level than that of the dry control and 5% pore former level. This suggested that the dry composite membrane became more elastic than dry control at higher pore former levels. In effect, the terpolymer softened the ethylcellulose without weakening the overall membrane.

The mechanical properties of polymer coatings in their wet state have tremendous impact on the mechanisms of drug release from polymer-coated dosage forms. For Surelease coating, several mechanisms are possible depending on the tensile strength and flexibility of the coating, including diffusion through a continuous polymer phase, diffusion through aqueous pores, and release driven by osmotic effects⁴³⁻⁴⁷.

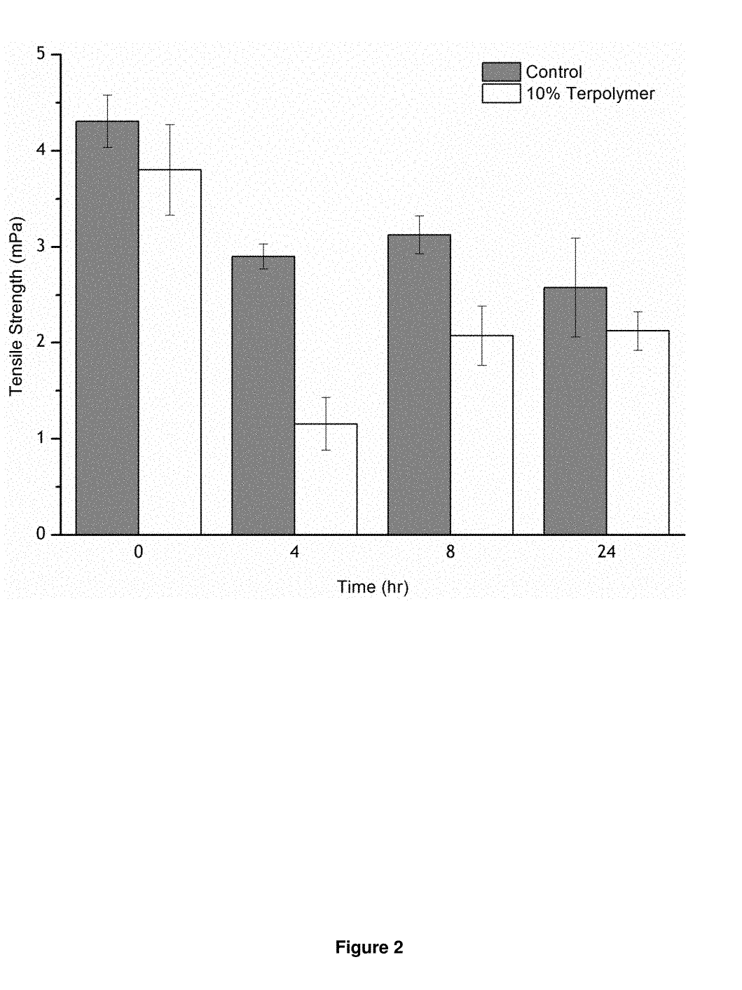

Both control and composite membranes at 10% pore former level have significantly lower tensile strength in their wet state compared to their dry state (FIG. 2). The wet composite membrane had drastically lower tensile strength, by 151%, compared to the wet control membrane after 4 hours in pH 6.8 phosphate buffer. However, tensile strength of the wet composite membrane improved over time and was only 21.3% lower after 24 hours. On the other hand, the tensile strength of the wet control membrane did not significantly change over 24 hours. The drastic decrease in tensile strength of the composite membrane may be due to the much faster water uptake of the terpolymer nanoparticles in comparison to the ethylcellulose. The sharp interface between the swollen nanoparticles and the relatively dry ethylcellulose bulk may have introduced additional internal stress in the membrane, thus weakening it. As the ethylcellulose swelled over time, the interface became less pronounce and thereby, relieving some of the internal stress of the membrane and increasing the tensile strength.

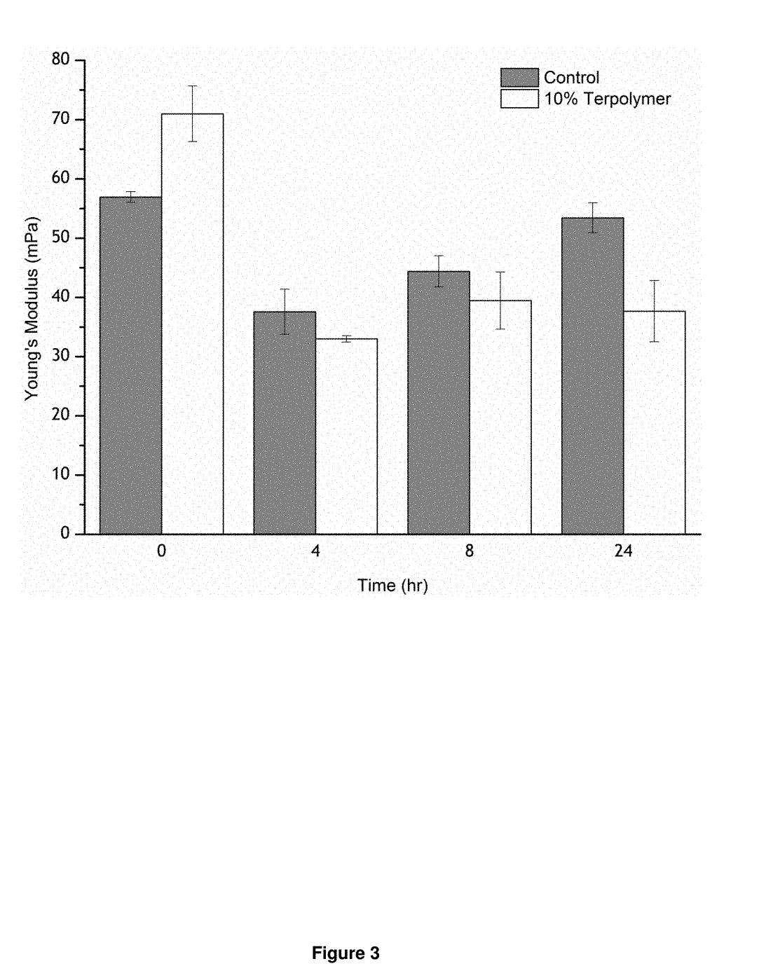

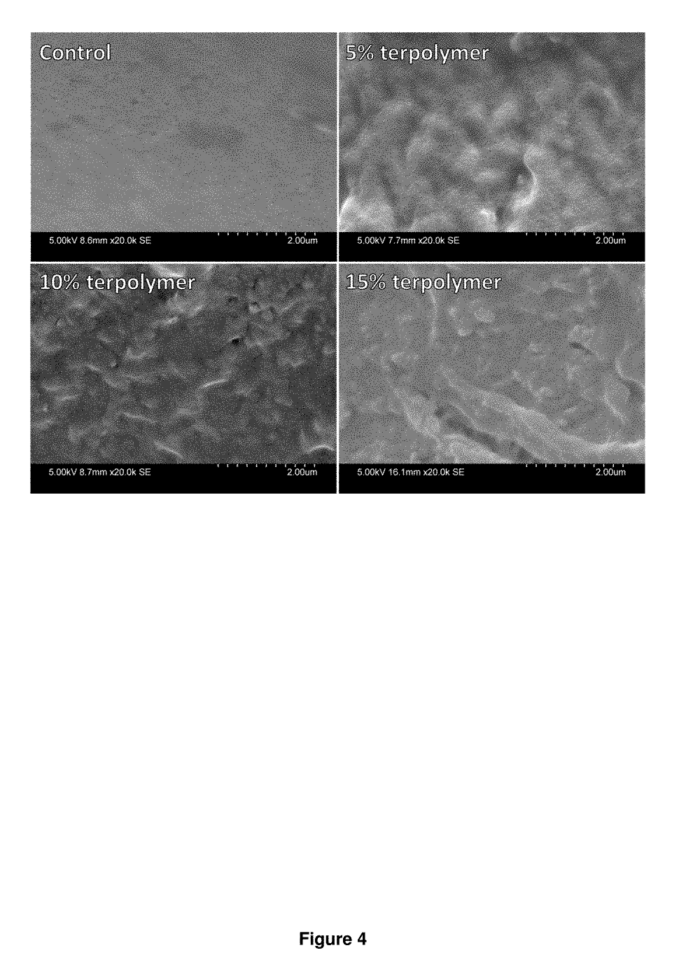

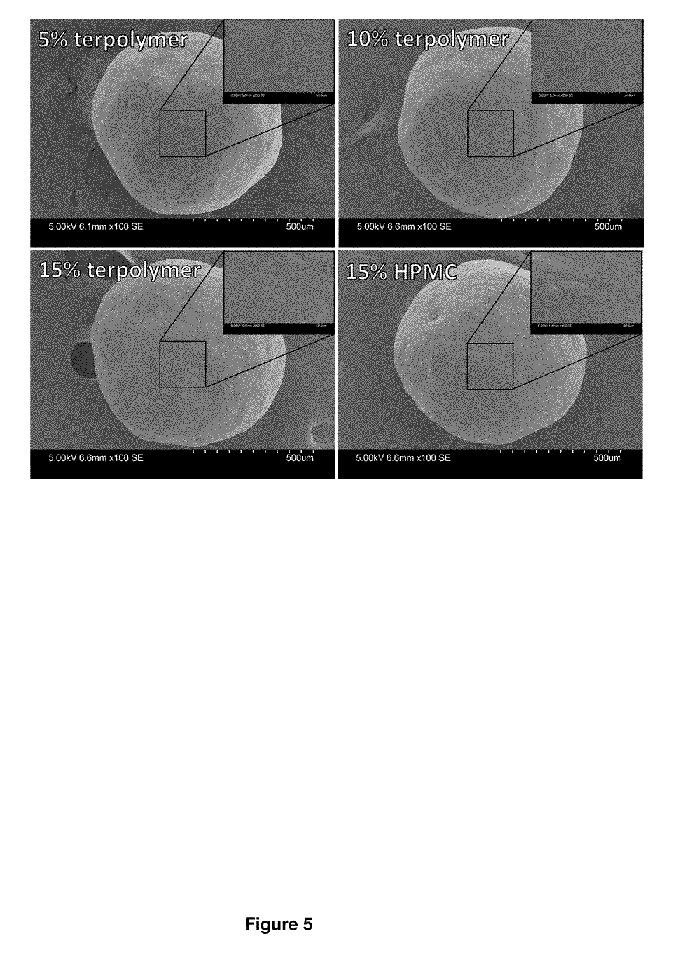

Young's modulus of the control membrane and composite membrane at 10% pore former level also significantly decreased in their wet state versus their dry state (FIG. 3). The increased flexibility of both control and composite membranes was likely caused by the plasticizing effect of water. Water can increase the motility of polymer chains by disrupting interchain interactions, especially hydrogen bonding. Young's modulus of the wet composite membrane did not significantly change over 24 hours, while slightly increasing for the wet control membrane.

T_{g} of Polymeric Composite Membrane:

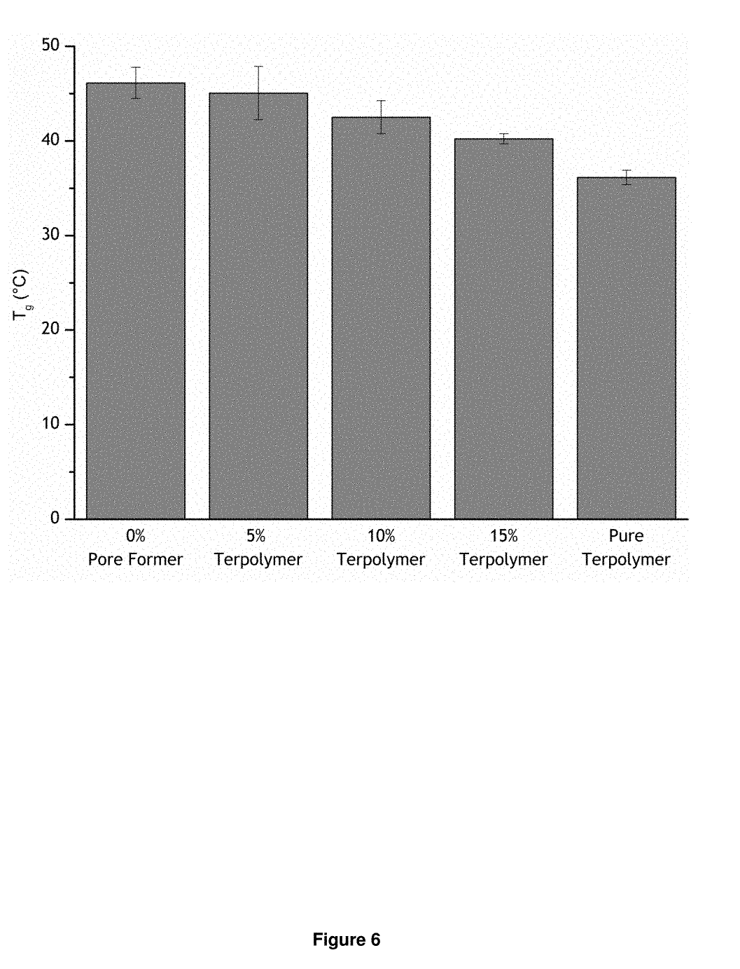

Increasing pore former level of terpolymer nanoparticles decreased the T_{g} of the composite membrane (FIG. 6). Although the T_{g} did not significantly decrease at 5% and 10% pore former levels, the difference in T_{g} became significant at 15% pore former level, lowering the T_{g} by approximately 6° C. The terpolymer nanoparticles may have a slight plasticizing effect on the Surelease membrane as the nanoparticles likely disrupted the packing of ethylcellulose chains. The PMAA of the terpolymer nanoparticles may have also disrupted any interchain hydrogen bonding between ethylcellulose chains.

Permeability of Polymeric Composite Membrane:

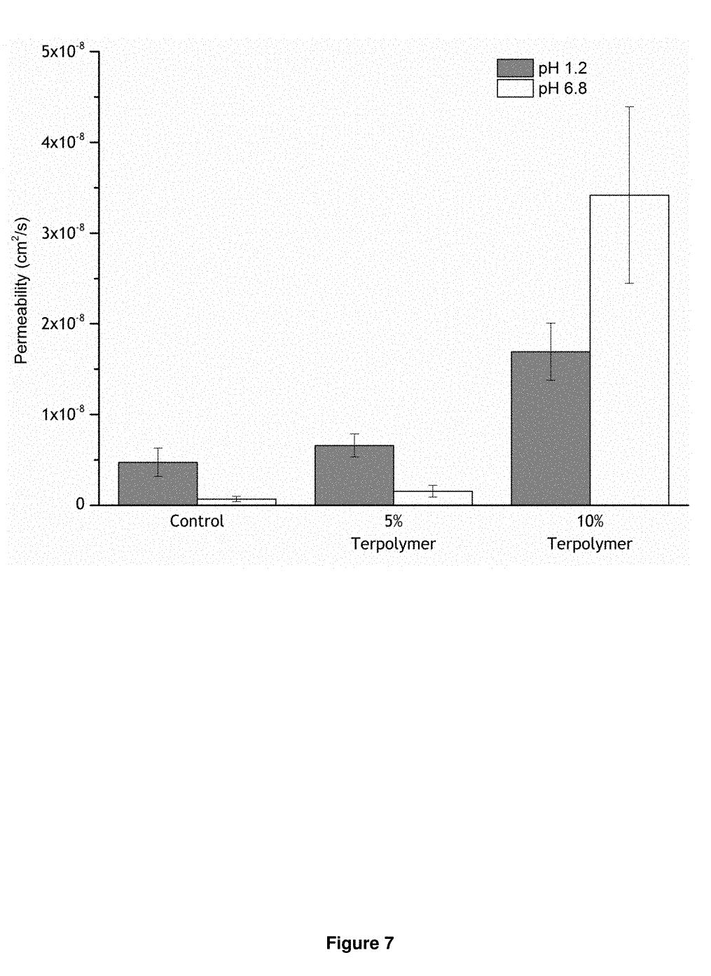

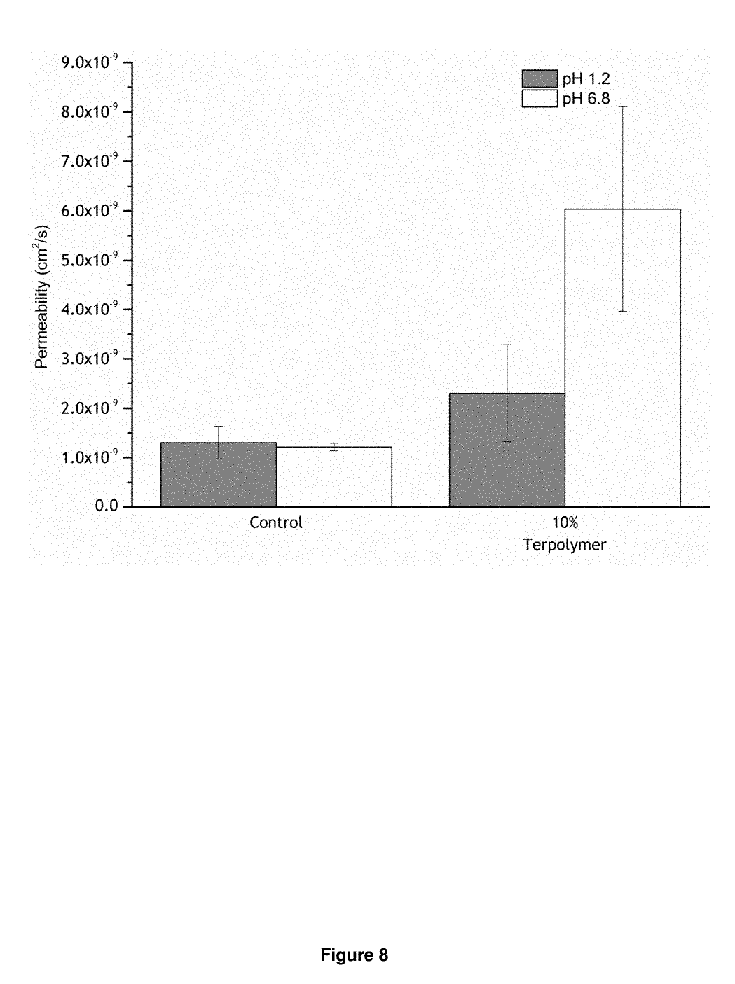

Permeabilities of the control and composite membranes were determined at pH 1.2 and 6.8 (FIG. 7). Verapamil HCl, a weakly basic drug with pH-dependent solubility, and theophylline, a neutral drug, were used as model drugs to compare the permeability at pH 1.2 and 6.8. For the control membrane and composite membrane at 5% pore former level, the permeability of verapamil HCl at pH 1.2 was significantly higher than at pH 6.8. However, the composite membrane at 10% pore former level exhibited significantly higher permeability of verapamil HCl at pH 6.8 than at pH 1.2 by over 2 folds. The permeability of theophylline showed no difference in permeability between pH 1.2 and 6.8 for the control, as expected (FIG. 8). On the other hand, for the composite membrane at 10% pore former level, the permeability of theophylline at pH 6.8 was over 2.6 fold higher than at pH 1.2. Furthermore, the permeability of diltiazem at pH 6.8 was over 40 fold higher than at pH 1.2 (FIG. 21). The result suggested that mechanism of permeation enhancement was likely through water flux induced by ionized PMAA chains of the nanoparticles, which caused the overall composite membrane to swell. The increased water content of the membrane likely induced formation of aqueous pores in which the drug can easily diffuse through.

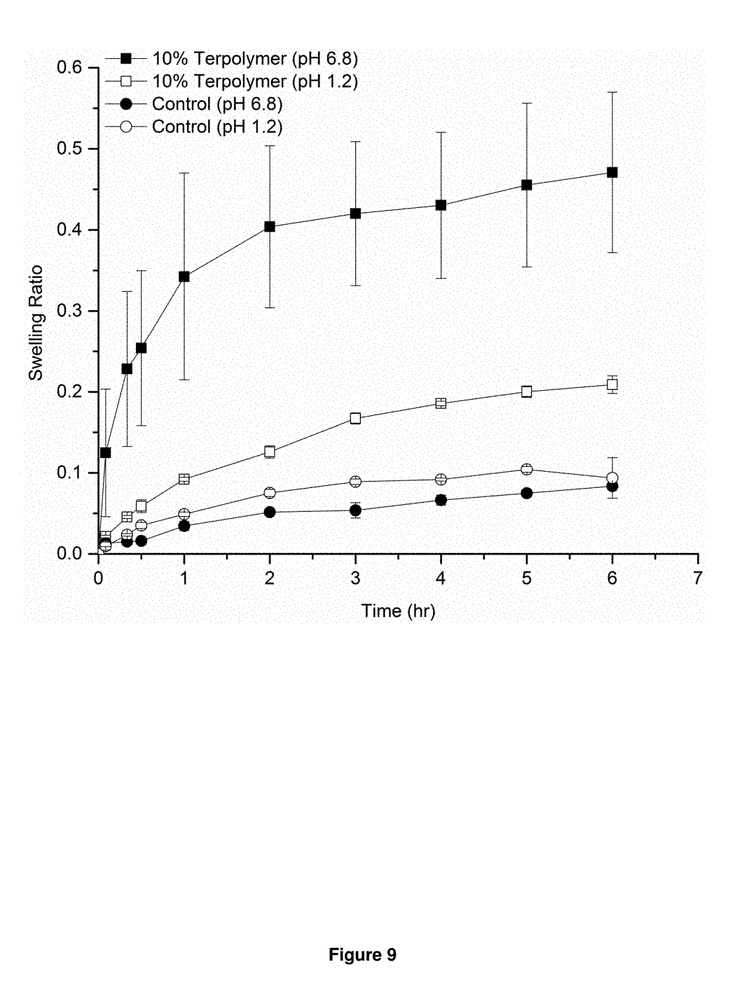

Swelling of polymeric composite membrane: To confirm that water flux were induced by ionization of PMAA of the terpolymer nanoparticle at higher pH, the swelling kinetics of the composite membrane at 10% pore former level were studied at pH 1.2 and 6.8. FIG. 9 shows swelling kinetics based on the weight of the membranes. Swelling of the membranes started to plateau at approximately 4 hours for the control and composite membranes. Control membrane had similar swelling at both pH 1.2 and 6.8. However, the composite membrane had significantly higher swelling ratio and swelling rate at pH 6.8 than at pH 1.2. At pH 6.8, the higher water content in the membrane increased the motility and free volume of the ethylcellulose polymer chain as well as induced the formation of water channels^{48,49}, where diffusion coefficient of the drug is much higher, in the polymer network. While at pH 1.2, the PMAA of the nanoparticles became unionized, leading to decreased water flux and lower permeability. The composite membrane functioned very similarly to a pH-responsive hydrogel⁵⁰⁻⁵². The composite membrane is ideal for the controlled release of weakly basic drug. In the stomach, the solubility of a weakly basic drug is high, however; in the small intestine the solubility of the drug would decrease due to the higher pH. To compensate for the low solubility in the small intestine, the increased permeability of the nanoparticle embedded membrane can achieve a steady release throughout the GI tract in a pH-independent manner.

Dissolution of Drug-Loaded Beads with Polymeric Composite Coating:

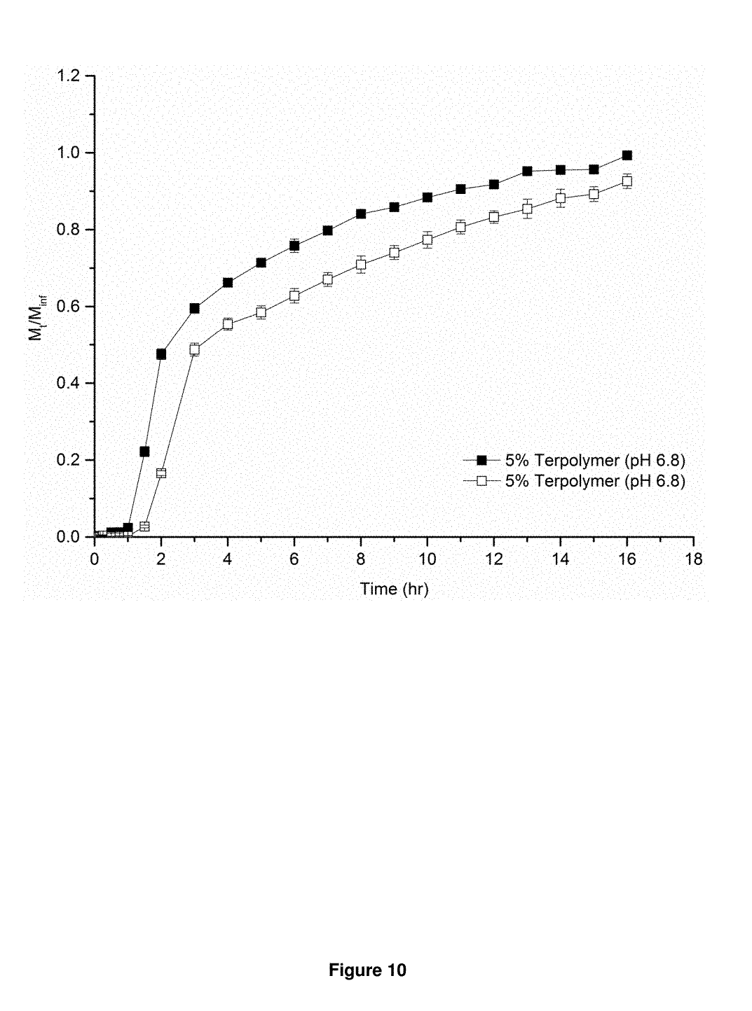

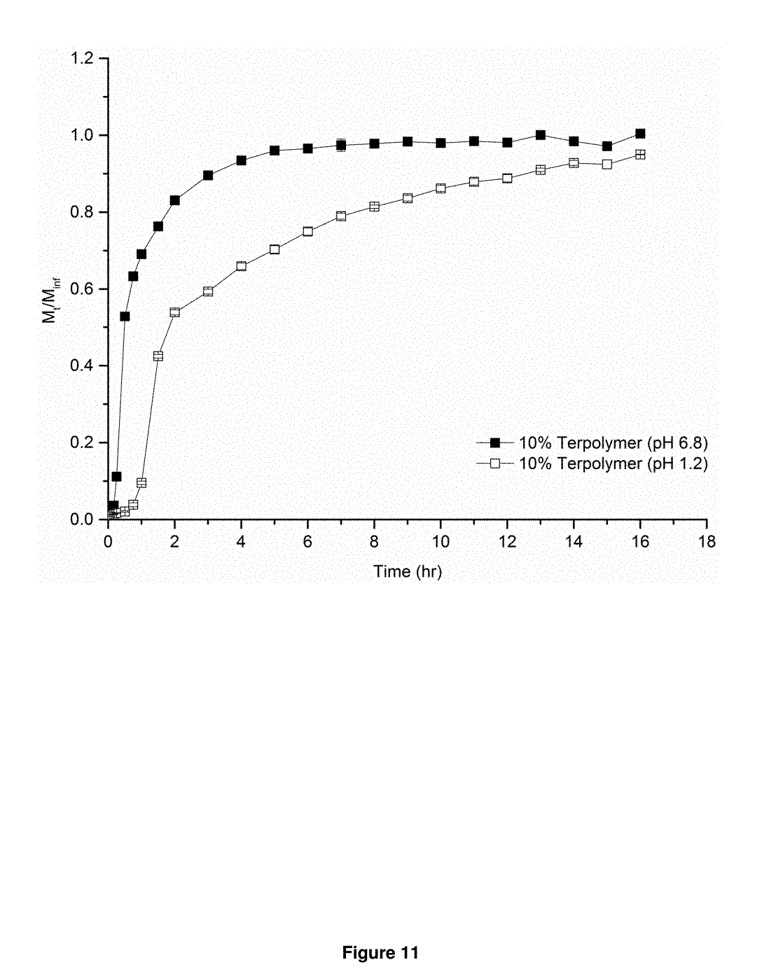

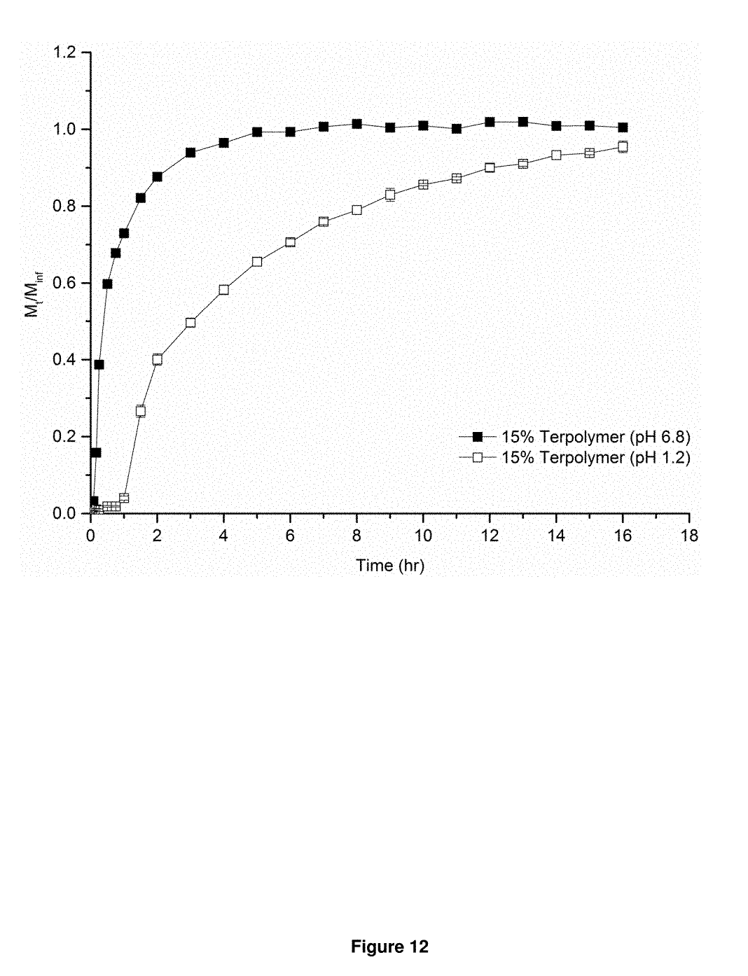

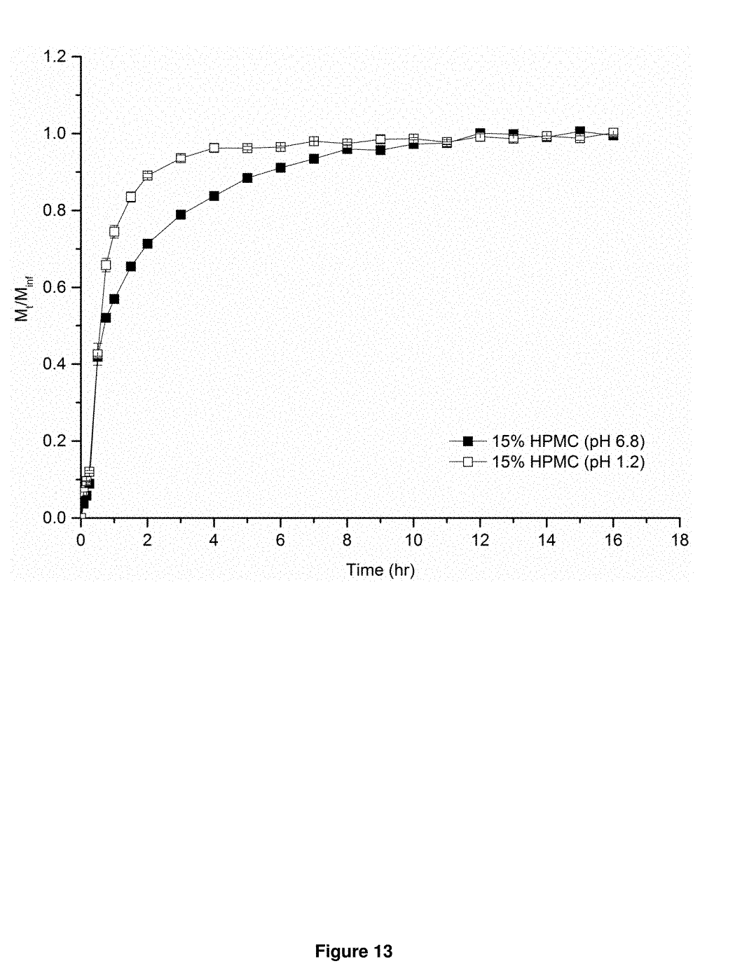

To further test the ability of the terpolymer nanoparticles to modulate the permeability of the polymeric composite coating, dissolution tests were conducted at pH 1.2 using 0.1 N HCl and at pH 6.8 using phosphate buffer. Diltiazem HCl was used as a model weakly basic drug due to its pH-dependent solubility. Even at low pore former level of 5%, the composite coating was able to sufficiently increase the coating permeability to compensate for the lower drug solubility at pH 6.8 (FIG. 10). The degree of permeability enhancement at pH 6.8 was further increased at higher pore former levels of 10% and 15% (FIGS. 11 and 12), which allows the composite coating to remain effective in achieving pH-independent release of drugs with low solubility at basic pH. However, 15% HPMC showed higher drug release at pH 1.2 than at pH 6.8 (FIG. 13), indicating a lack of permeability enhancement by HPMC required to compensate for the decrease in drug solubility at higher pH. The composite coating is an elegant and effective approach in achieving pH-independent controlled release of weakly basic drugs due to its simplicity and flexibility of enhancing coating permeability in response to changes in pH.

pH-Sensitivity of PDEAEM-g-Starch Nanoparticles:

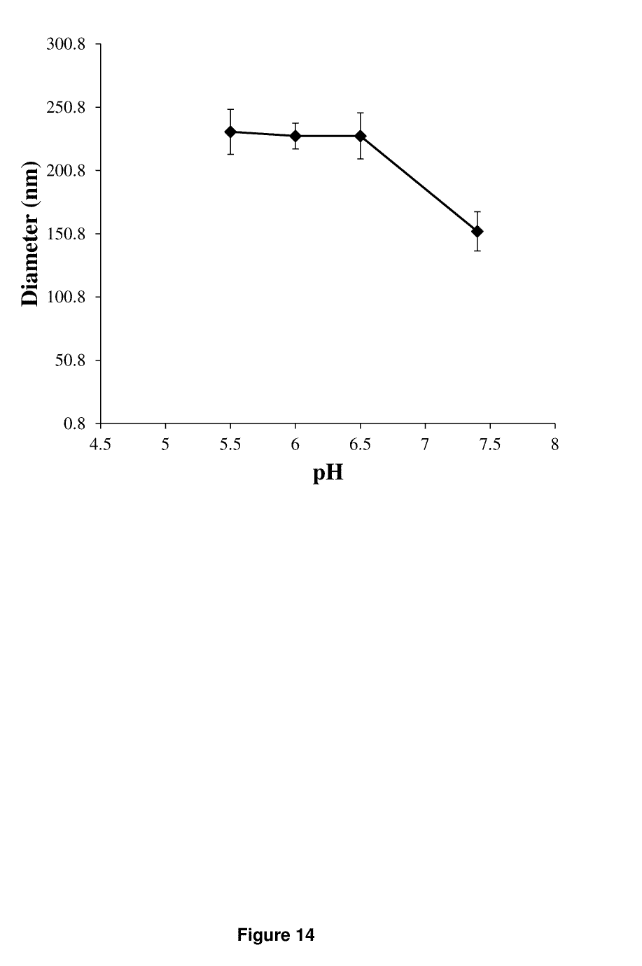

The pH-responsiveness of PDEAEM-g-starch nanoparticles is shown in FIG. 14. The nanoparticles shrunk in high pH and swelled in low pH with a 44.5-59.5% increase in diameter from pH 7.4 to 5.5.

Morphology of PDEAEM-g-Starch Nanoparticle-Embedded Ethylcellulose Composite Membrane:

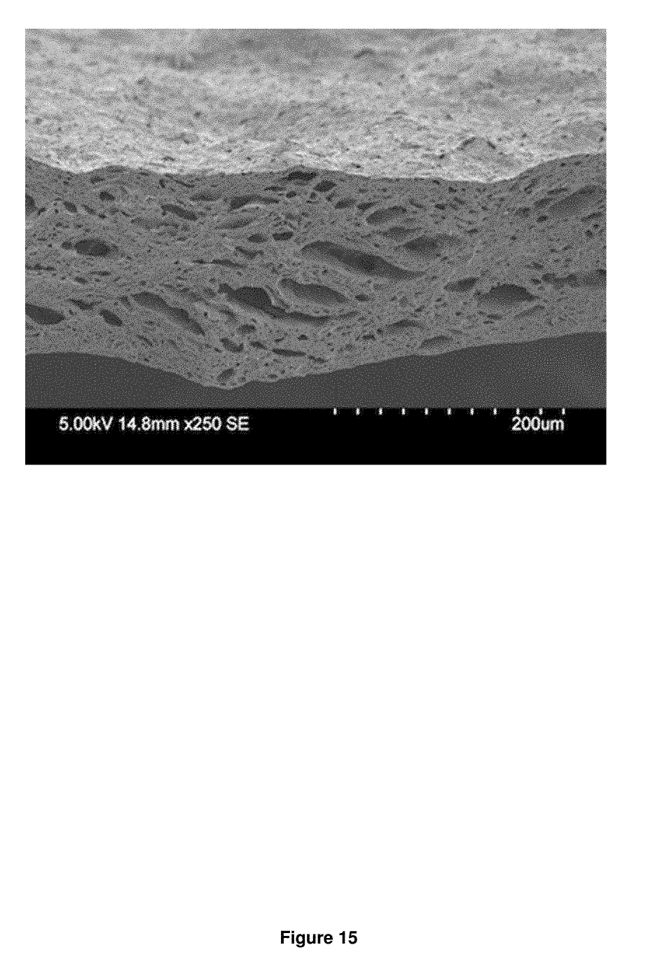

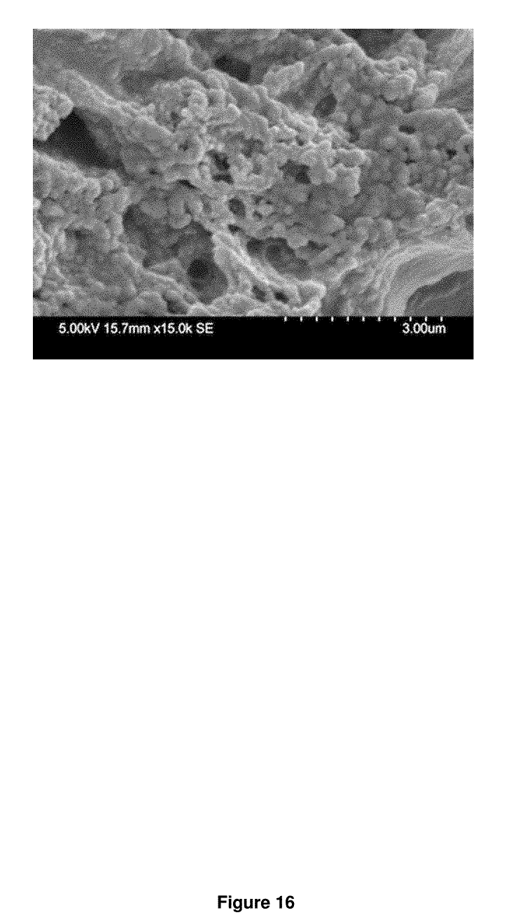

As portrayed in SEM phtographs (FIGS. 15-16), PDEAEM-g-starch nanoparticles mixed well with ethylcellulose to form a composite membrane. The membrane was porous when dried.

Permeability of PDEAEM-g-Starch Nanoparticle-Embedded Ethylcellulose Composite Membrane:

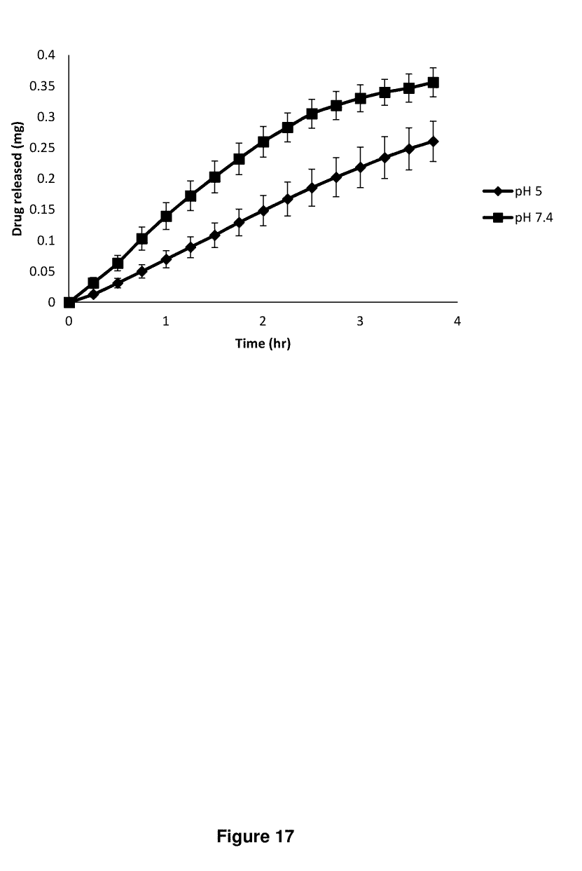

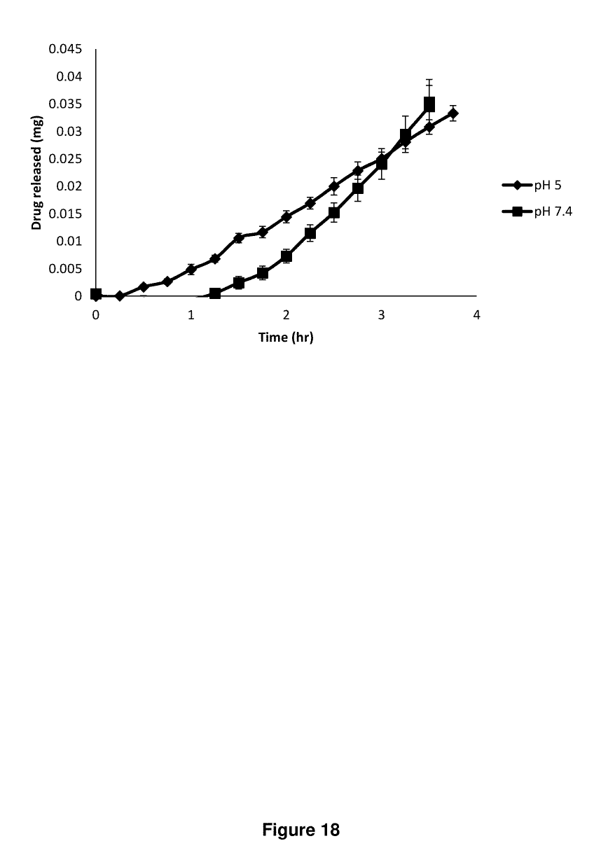

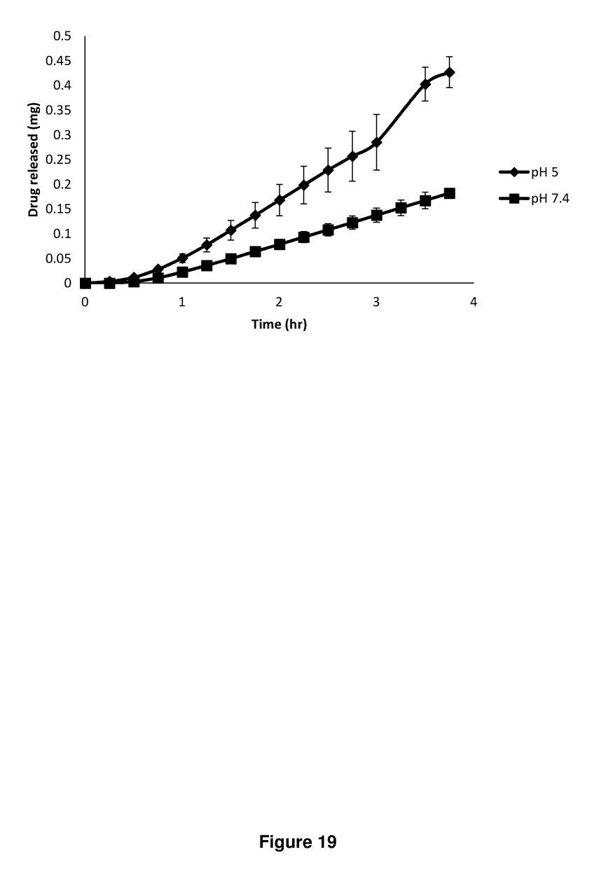

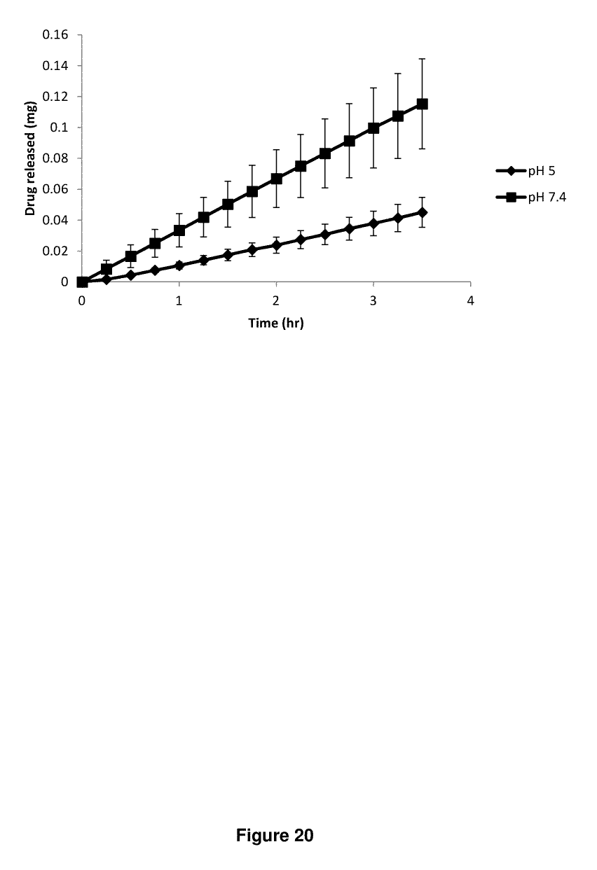

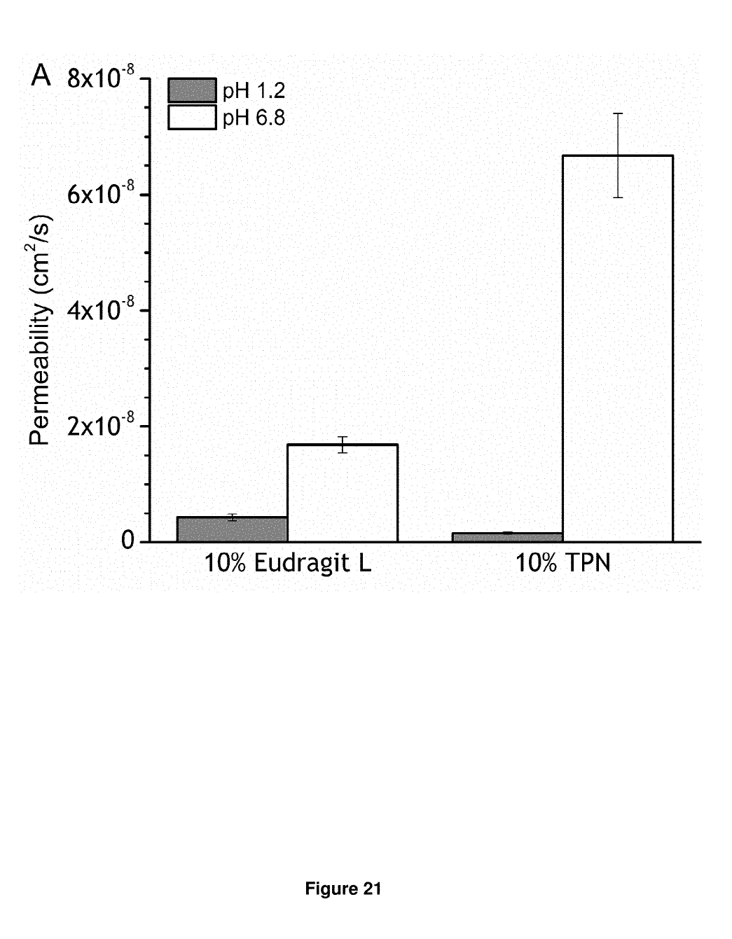

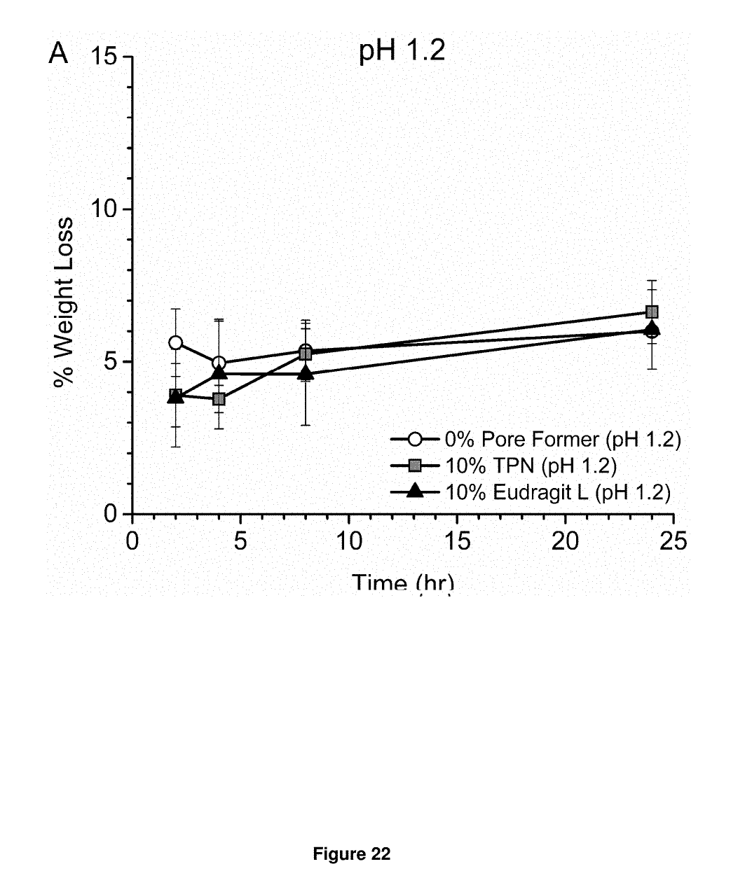

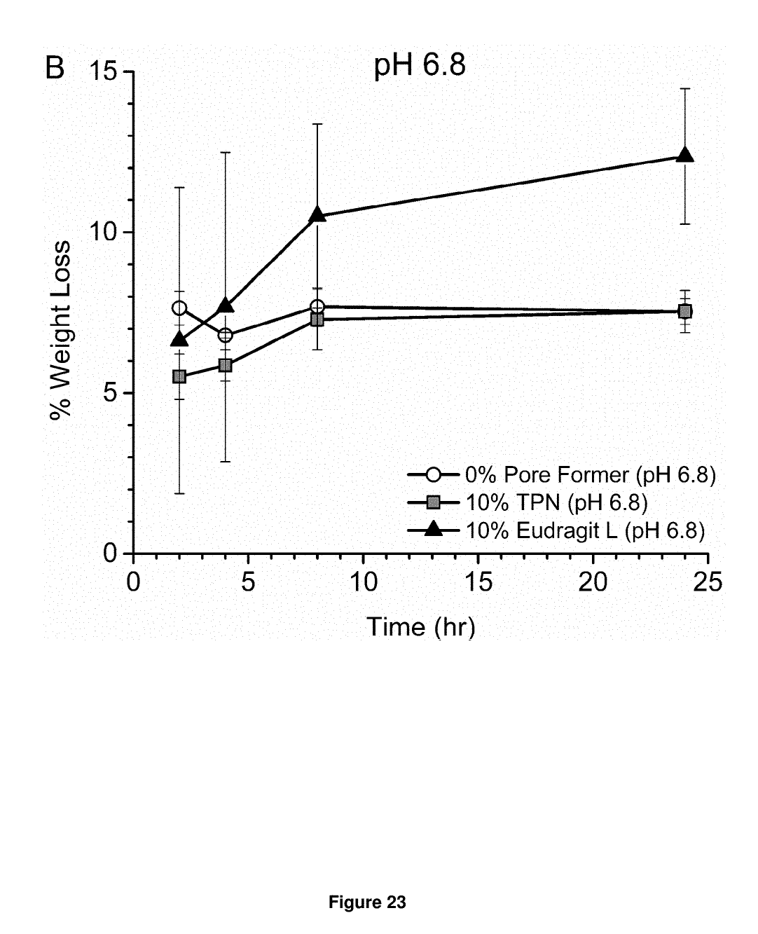

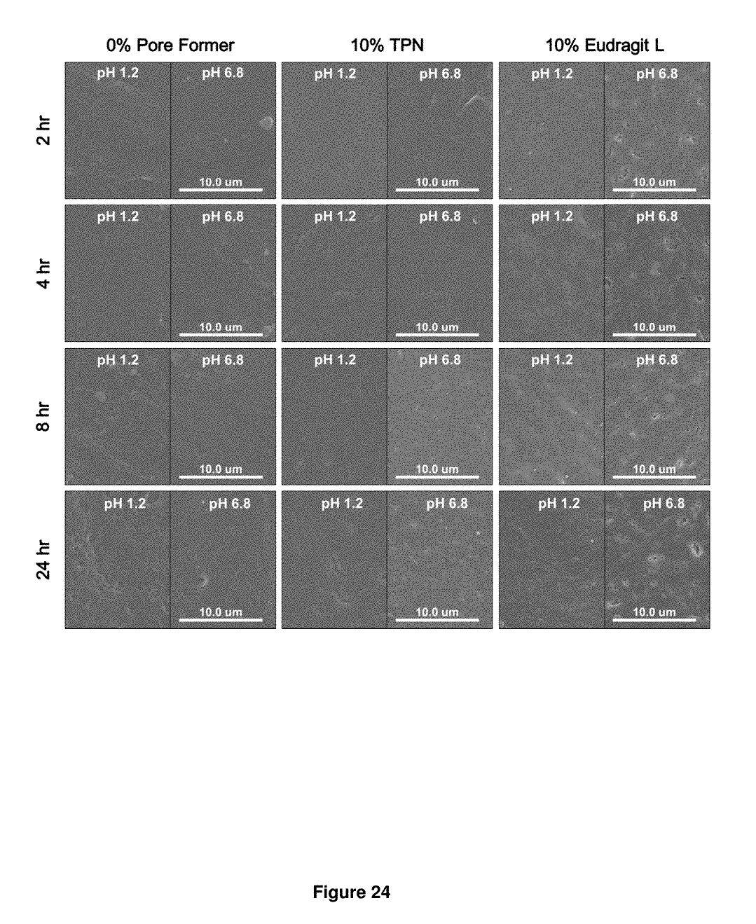

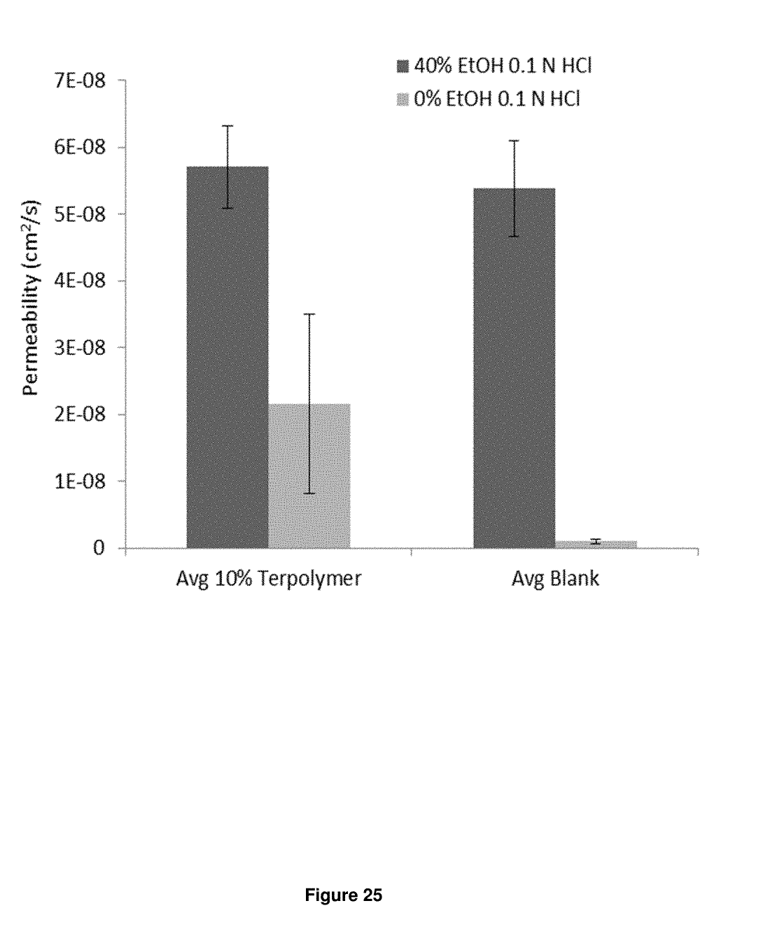

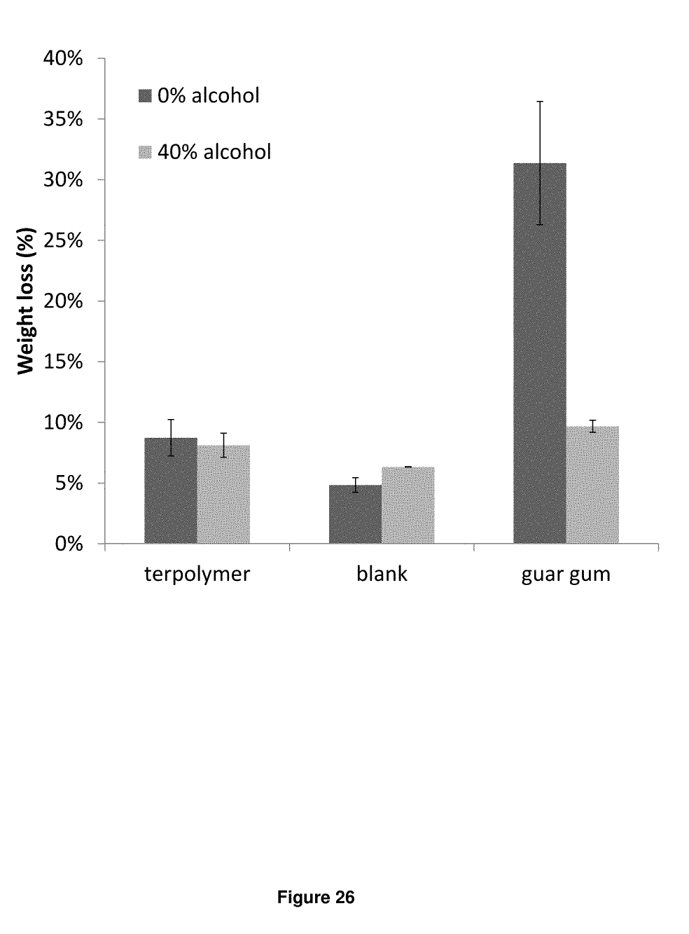

Drug permeation kinetics at pH 7.4 and pH 5 across the composite membrane are shown in FIGS. 17-20. Drug release rate of non-ionic drug theophylline showed a strong pH dependence. It can be seen that there is a substantial increase in permeability with pH for theophylline (FIG. 17) and verapamil (FIG. 18), as expected from theory. However, release rate of ibuprofen substantially decreases in permeability (FIG. 19). This could be a result of interactions between the membrane and ibuprofen that could have altered the properties and/or integrity of the membrane. Release rate of vitamin B12 exhibited a pH dependence (FIG. 20). A larger proportion of drug permeated at pH 7.4 than at pH 5.5 for vitamin B12 compared to theophylline, likely due to the larger molecular size of vitamin B12.

Leaching of Pore Formers from Polymeric Composite Coating:

The weight loss of blank, polymeric composite (10% TPN), and 10% Eudragit® L films were measured to determine the extent of leaching of pore formers from films at pH 1.2 (FIG. 22) and 6.8 (FIG. 23). After 24 hour-immersion in the pH 1.2 medium, both 10% TPN and 10% Eudragit® L films had very similar weight loss (˜6%) to that of blank films, likely due to water soluble excipients in the EC dispersion product. Since the acrylic acids in Eudragit® L and TPNs were unionized at pH 1.2, both pore formers were insoluble and leaching at pH 1.2 was negligible. At pH 6.8, both blank and 10% TPN-EC films experienced slight weight loss up to 24 hours similar to the films at pH 1.2; whereas 10% Eudragit® L films lost weight rapidly after 4 hours and reached 61% higher weight loss than blank and 10% TPN films after 24 hours. To examine whether the weight loss was correlated to the porosity and integrity of the films, SEM photographs of the films were acquired after the weight loss study. All three types of films showed negligible morphological change after immersing in pH 1.2 medium for up to 24 hours (FIG. 24), which was consistent with the weight loss results. After immersed in pH 6.8 phosphate buffer up to 24 hours, blank films showed no discernable signs of change in porosity (FIG. 24), similar to the films immersed at pH 1.2. The 10% TPN films only showed a few small pores after 8 hours, while 10% Eudragit® L films already showed substantial erosions and large pores at 2 hours. These results from the weight loss study and the subsequent SEM examination of the films clearly demonstrated that TPNs were able to mitigate leaching from EC films at both pH 1.2 and 6.8. The large size (˜240 nm in mean diameter) and insolubility rendered by crosslinking of TPNs made them difficult to migrate out of the EC films. At 10% pore former level, lower than the percolation threshold (i.e. 20-30 wt %)⁵³, no interconnected permeable channels could be formed that allow the TPNs to diffuse out either. Moreover, the similarity of TPNs and EC in chain structure and hydrophobic domains could have imparted their high affinity, further maintaining TPNs within the EC mesh. In contrast, Eudragit® L, as a soluble polymer at pH 6.8, showed significant leaching after 2 hours which would weaken the mechanical strength of the films.

Permeabilities of theophylline across the blank and composite membranes were determined at in both 0% or 40% ethanol concentration 0.1 N HCl (FIG. 25). The permeability of theophylline showed a great difference between 0% and 40% ethanol concentration for blank membrane at almost 51 folds, as expected as ethylcellulose is soluble in ethanol. On the other hand, for the composite membrane at 10% pore former level, the permeability of theophylline at 0% and 40% only increased 2.6 folds. The result suggested that the composite membrane is alcohol resistant in comparison to blank membrane. FIGS. 26 and 27 respectively exhibited much less weight loss and medium uptake for the terpolymer-containing composite membranes than the blank membranes. SEM images (FIG. 8) showed that the terpolymer-containing membrane was more intact after immersed in 40% alcohol than the blank membrane and guar gum-containing membrane.

In one aspect of the disclosure, a novel pH-responsive polymeric composite membrane was successfully synthesized by incorporating terpolymer nanoparticles into ethylcellulose coating. The new composite coating was homogenous and exhibited good mechanical properties similar to that of ethylcellulose coating with no pore former. The good mechanical properties, together with the low impact on viscosity of the coating dispersion and the pH-responsive permeability, make the composite coating a good candidate for controlled release formulation and other pharmaceutical applications. We were also able to demonstrate pH-independent release of diltiazem HCl from drug-loaded beads coated with composite coating. Additionally, the terpolymer showed very low solubility in ethanolic solutions, from which an alcohol-resistant composite membrane was successfully synthesized.

Generally speaking, the polymeric composite coating and methods herein are for controlled release of ingredients in pharmaceutical formulations, nutraceuticals, animal care products, and consumer products. Various embodiments and aspects of the disclosure have been described with reference to details discussed above. The description and drawings are illustrative of the disclosure and are not to be construed as limiting the disclosure. Numerous specific details have been described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “approximately” is meant to cover slight variations that may exist in the upper and lower limits so as to not exclude embodiments where on average most of the dimensions are satisfied but where statistically dimensions may exist outside this region. It is not the intention to exclude embodiments such as these from the present disclosure.

### REFERENCES

1 Prasertmanakit, S., Praphairaksit, N., Chiangthong, W. & Muangsin, N. Ethyl cellulose microcapsules for protecting and controlled release of folic acid. AAPS PharmSciTech 10, 1104-1112, doi:10.1208/s12249-009-9305-3 (2009).
2 Dyke, D. G. Pore forming sterilization bag. U.S. Pat. No. 4,515,841 (1985).
3 Sugawara, M. et al. The use of an in vitro dissolution and absorption system to evaluate oral absorption of two weak bases in pH-independent controlled-release formulations. European Journal of Pharmaceutical Sciences 26, 1-8, doi:10.1016/j.ejps.2005.02.017 (2005).
4 Thoma, K. & Zimmer, T. Retardation of weakly basic drugs with diffusion tablets. International Journal of Pharmaceutics 58, 197-202 (1990).
5 Thoma, K. & Ziegler, I. The pH-independent release of fenoldopam from pellets with insoluble film coats. European Journal of Pharmaceutics and Biopharmaceutics 46, 105-113 (1998).
6 Akiyama, Y. et al. pH-independent controlled-release microspheres using polyglycerol esters of fatty acids. Journal of Pharmaceutical Sciences 83, 1600-1607 (1994).
7 Rao, V. M., Engh, K. & Qiu, Y. Design of pH-independent controlled release matrix tablets for acidic drugs. International Journal of Pharmaceutics 252, 81-86, doi:http://dx.doi.org/10.1016/S0378-5173(02)00622-1 (2003).
8 Tatavarti, A. S. & Hoag, S. W. Microenvironmental pH modulation based release enhancement of a weakly basic drug from hydrophilic matrices. Journal of Pharmaceutical Sciences 95, 1459-1468 (2006).
9 Kohri, N. et al. Evaluation of pH-independent sustained-release granules of dipyridamole by using gastric-acidity-controlled rabbits and human subjects. International Journal of Pharmaceutics 81, 49-58, doi:http://dx.doi.org/10.1016/0378-5173(92)90042-Z (1992).
10 Oren, P. L. & Seidler, W. M. K. Sustained release matrix. U.S. Pat. No. 4,968,508 (1990).
11 Howard, J. R. & Timmins, P. Controlled release formulation. U.S. Pat. No. 4,792,452 (1988).
12 Zentner, G. M., Rork, G. S. & Himmelstein, K. J. Osmotic flow through controlled porosity films: An approach to delivery of water soluble compounds. Journal of Controlled Release 2, 217-229, doi:http://dx.doi.org/10.1016/0168-3659(85)90047-1 (1985).
13 Lecomte, F., Siepmann, J., Walther, M., MacRae, R. J. & Bodmeier, R. Blends of enteric and GIT-insoluble polymers used for film coating: physicochemical characterization and drug release patterns. Journal of Controlled Release 89, 457-471, doi:https://doi.org/10.1016/S0168-3659(03)00155-X (2003).
14 Sakellariou, P., Rowe, R. C. & White, E. F. T. Polymer/polymer interaction in blends of ethyl cellulose with both cellulose derivatives and polyethylene glycol 6000. International Journal of Pharmaceutics 34, 93-103, doi:https://doi.org/10.1016/0378-5173(86)90014-1 (1986).
15 Sakellariou, P. & Rowe, R. C. Phase separation and morphology in ethylcellulose/cellulose acetate phthalate blends. Journal of Applied Polymer Science 43, 845-855, doi:10.1002/app.1991.070430502 (1991).
16 Lecomte, F., Siepmann, J., Walther, M., MacRae, R. J. & Bodmeier, R. pH-Sensitive polymer blends used as coating materials to control drug release from spherical beads: elucidation of the underlying mass transport mechanisms. Pharmaceutical research 22, 1129-1141, doi:10.1007/s11095-005-5421-2 (2005).
17 Khan, M. Z. I., Prebeg, Z̆. & Kurjaković, N. A pH-dependent colon targeted oral drug delivery system using methacrylic acid copolymers: I. Manipulation of drug release using Eudragit® L100-55 and Eudragit® S100 combinations. Journal of Controlled Release 58, 215-222, doi:https://doi.org/10.1016/S0168-3659(98)00151-5 (1999).
18 Akiyama, Y. et al. pH-Independent Controlled-Release Microspheres Using Polyglycerol Esters of Fatty Acids. Journal of Pharmaceutical Sciences 83, 1600-1607, doi:10.1002/jps.2600831116 (2010).
19 Howard, J. R. & Timmins, P. (Google Patents, 1988).
20 Amighi, K., Timmermans, J., Puigdevall, J., Baltes, E. & Moës, A. J. Peroral Sustained-Release Film-Coated Pellets as a Means to Overcome Physicochemical and Biological Drug-Related Problems. I. In Vitro Development and Evaluation. Drug Development and Industrial Pharmacy 24, 509-515, doi:10.3109/03639049809085651 (1998).
21 R. B. P. Gruber, G. B., H. Stricker. Dipyridamole sustained release forms comprising lacquer-coated particles and the preparation thereof, in, U.S. (1983).
22 Cole, E. T. et al. Enteric coated HPMC capsules designed to achieve intestinal targeting. International Journal of Pharmaceutics 231, 83-95, doi:https://doi.org/10.1016/S0378-5173(01)00871-7 (2002).
23 Felton, L. A. & Porter, S. C. An update on pharmaceutical film coating for drug delivery. Expert opinion on drug delivery 10, 421-435, doi:10.1517/17425247.2013.763792 (2013).
24 Siepmann, F., Siepmann, J., Walther, M., MacRae, R. J. & Bodmeier, R. Polymer blends for controlled release coatings. Journal of Controlled Release 125, 1-15, doi:https://doi.org/10.1016/j.jconre1.2007.09.012 (2008).
25 Siepmann, F., Siepmann, J., Walther, M., MacRae, R. J. & Bodmeier, R. Blends of aqueous polymer dispersions used for pellet coating: Importance of the particle size. Journal of Controlled Release 105, 226-239, doi:https://doi.org/10.1016/j.jconre1.2005.03.028 (2005).
26 Parikh, N. H., Porter, S. C. & Rohera, B. D. Tensile properties of free films cast from aqueous ethylcellulose dispersions. Pharmaceutical research 10, 810-815 (1993).
27 Bussemer, T., Peppas, N. A. & Bodmeier, R. Time-Dependent Mechanical Properties of Polymeric Coatings Used in Rupturable Pulsatile Release Dosage Forms. Drug Development and Industrial Pharmacy 29, 623-630, doi:10.1081/DDC-120021311 (2003).
28 Ensslin, S., Moll, K. P., Haefele-Racin, T. & Mader, K. Safety and robustness of coated pellets: self-healing film properties and storage stability. Pharmaceutical research 26, 1534-1543, doi:10.1007/s11095-009-9866-6 (2009).
29 Bose, S. & Bogner, R. H. Solventless Pharmaceutical Coating Processes: A Review. Pharmaceutical development and technology 12, 115-131, doi:10.1080/10837450701212479 (2007).
30 Cerea, M., Zheng, W., Young, C. R. & McGinity, J. W. A novel powder coating process for attaining taste masking and moisture protective films applied to tablets. International Journal of Pharmaceutics 279, 127-139, doi:https://doi.org/10.1016/j.ijpharm.2004.04.015 (2004).
31 Chaney, M. A. Side effects of intrathecal and epidural opioids. Canadian journal of anaesthesia=Journal canadien d'anesthesie 42, 891-903, doi:10.1007/bf03011037 (1995).
32 Jedinger, N., Khinast, J. & Roblegg, E. The design of controlled-release formulations resistant to alcohol-induced dose dumping—A review. European Journal of Pharmaceutics and Biopharmaceutics 87, 217-226, doi:http://dx.doi.org/10.1016/j.ejpb.2014.02.008 (2014).
33 FDA. FDA Alert for Healthcare Professionals: Hydromorphone Hydrochloride Extended-release Capsules (marketed as Palladone), <https://www.fda.gov/Drugs/DrugSafety/ucm129288.htm> (2005).
34 Franke, A., Teyssen, S., Harder, H. & Singer, M. V. Effect of ethanol and some alcoholic beverages on gastric emptying in humans. Scandinavian journal of gastroenterology 39, 638-644, doi:10.1080/00365520410005009 (2004).
35 Franke, A., Nakchbandi, I. A., Schneider, A., Harder, H. & Singer, M. V. The effect of ethanol and alcoholic beverages on gastric emptying of solid meals in humans. Alcohol and alcoholism (Oxford, Oxfordshire) 40, 187-193, doi:10.1093/alcalc/agh138 (2005).
36 Bujanda, L. The effects of alcohol consumption upon the gastrointestinal tract. Am J Gastroenterol 95, 3374-3382 (2000).
37 Ohara, T., Kitamura, S., Kitagawa, T. & Terada, K. Dissolution mechanism of poorly water-soluble drug from extended release solid dispersion system with ethylcellulose and hydroxypropylmethylcellulose. International Journal of Pharmaceutics 302, 95-102, doi:https://doi.org/10.1016/j.ijpharm.2005.06.019 (2005).
38 Gohel, M. & Bariya, S. H. Advanced formulation design of venlafaxine hydrochloride coated and triple-layer tablets containing hypromellose. Pharmaceutical development and technology 14, 650-658, doi:10.3109/10837450902911911 (2009).
39 Rosiaux, Y. et al. Ethanol-resistant ethylcellulose/guar gum coatings—Importance of formulation parameters. European Journal of Pharmaceutics and Biopharmaceutics 85, 1250-1258, doi:http://dx.doi.org/10.1016/j.ejpb.2013.07.014 (2013).
40 Rosiaux, Y. et al. Mechanisms controlling theophylline release from ethanol-resistant coated pellets. Pharmaceutical research 31, 731-741, doi:10.1007/s11095-013-1194-1 (2014).
41 Jedinger, N. et al. Alcohol dose dumping: The influence of ethanol on hot-melt extruded pellets comprising solid lipids. European Journal of Pharmaceutics and Biopharmaceutics 92, 83-95, doi:10.1016/j.ejpb.2015.02.022 (2015).
42 Jedinger, N., Schrank, S., Fischer, J. M. et al. Development of an Abuse- and Alcohol-Resistant Formulation Based on Hot-Melt Extrusion and Film Coating. AAPS PharmSciTech 17, doi:doi:10.1208/s12249-015-0373-2 (2016).
43 Bodmeier, R. & Paeratakul, O. Mechanical Properties of Dry and Wet Cellulosic and Acrylic Films Prepared from Aqueous Colloidal Polymer Dispersions Used in the Coating of Solid Dosage Forms. Pharmaceutical Research: An Official Journal of the American Association of Pharmaceutical Scientists 11, 882-888, doi:10.1023/a:1018942127524 (1994).
44 Husson, I., Leclerc, B., Spenlehauer, G., Veillard, M. & Couarraze, G. Modelling of drug release from pellets coated with an insoluble polymeric membrane. Journal of Controlled Release 17, 163-173, doi:10.1016/0168-3659(91)90056-j (1991).
45 Iyer, U., Hong, W. H., Das, N. & Ghebre-Sellassie, I. Comparative evaluation of three organic solvent and dispersion-based ethylcellulose coating formulations. Pharm. Technol. 14, 68-86 (1990).
46 Ozturk, A. G., Ozturk, S. S., Palsson, B. O., Wheatley, T. A. & Dressman, J. B. Mechanism of release from pellets coated with an ethylcellulose-based film. Journal of Controlled Release 14, 203-213, doi:10.1016/0168-3659(90)90160-u (1990).
47 Zhang, G., Schwartz, J. B. & Schnaare, R. L. Bead Coating. I. Change in Release Kinetics (and Mechanism) Due to Coating Levels. Pharmaceutical Research: An Official Journal of the American Association of Pharmaceutical Scientists 8, 331-335, doi:10.1023/a:1015837330463 (1991).
48 Wagner, K. G. & Gruetzmann, R. Anion-induced water flux as drug release mechanism through cationic Eudragit RS 30D film coatings. The AAPS journal [electronic resource]. 7, E668-677 (2005).
49 Fda, U. S. Guidance for industry: Extended release oral dosage forms, development, evaluation, and application of in vitro/in vivo correlations. Center for Drug Evaluation and Research, doi:Export Date 17 Aug. 2014 (1997).
50 Sutter, B. K. & Lippold, B. C. Parameters controlling drug release from pellets coated with aqueous ethyl cellulose dispersion. International Journal of Pharmaceutics 54, 15-25 (1989).
51 Bindschaedler, C., Gurny, R. & Doelker, E. Osmotic water transport through cellulose acetate membranes produced from a latex system. Journal of Pharmaceutical Sciences 76, 455-460 (1987).
52 Lippold, B. C., Lippold, B. H. & Lichey, J. Drug transport through lipophilic membranes. 3. Relationship between diffusion rates of drugs through membranes and their properties. Pharmazeutische Industrie 47, 1195-1201 (1985).
53 Zhang, K. & Wu, X. Y. Temperature and pH-responsive polymeric composite membranes for controlled delivery of proteins and peptides. Biomaterials 25, 5281-5291 (2004).

1. A polymeric composite coating comprising a drug release retardant polymer matrix, and a pH-responsive nanoparticulate pore former.

2. The polymeric composite coating of claim 1, wherein the drug release retardant polymer matrix comprises any one or a combination of cellulose derivatives, (alkyl) acrylate polymers and derivatives, polyvinyls and copolymers.

3. The polymeric composite coating of claim 1, wherein the pH-responsive nanoparticulate pore formers comprise a first polymer grafted to a second polymer, which is covalently bound to a third polymer.

4. The polymeric composite coating of claim 3, wherein the first polymer comprises a polysaccharide; the second polymer is a crosslinked polymer comprising of a ionizable polymer; and the third polymer is a polysorbate comprising a (C9-C31)R—C(O)O— group covalently bound to the second polymer by a C—C bond between the carbon backbone of the second polymer and the R group.

5. The polymeric composite coating of claim 4, wherein the ionizable polymer is any one of polymethacrylic acid, polyacrylic acid, and maleic acid copolymers, and polyvinyl derivatives.

6. The polymeric composite coating of claim 4, wherein the ionizable polymer is selected from methacrylic acid-ethacrylate copolymer, poly(2-(dimethylamino)ethyl methacrylate), poly(2-(diisopropylamino)ethyl methacrylate), poly(2-n-morpholinoethyl methacrylate), poly(1-vinylimidazole), poly(2-vinylpyridine), and (4-vinylpyridine).

7. The polymeric composite coating of claim 2, wherein the pH-responsive nanoparticulate pore formers comprise a first polymer grafted to a second polymer, which is covalently bound to a third polymer.

8. The polymeric composite coating of claim 7, wherein the first polymer comprises a polysaccharide; the second polymer is a crosslinked polymer comprising of an ionizable polymer grafted to the first polymer; and the third polymer is a polysorbate comprising a (C9-C31)R—C(O)O— group covalently bound to the second polymer by a C—C bond between the carbon backbone of the second polymer and the R group.

9. The polymeric composite coating of claim 8, wherein the ionizable polymer is any one of polymethacrylic acid derivatives, acrylic acid derivatives, maleic acid copolymers, and polyvinyl derivatives.

10. The polymeric composite coating of claim 8, wherein the ionizable polymer is selected from poly(methacrylic acid), poly(acrylic acid), methacrylic acid-methacrylate copolymer, methacrylic acid-ethacrylate copolymer, poly(2-(dimethylamino)ethyl methacrylate), poly(2-(diisopropylamino)ethyl methacrylate), poly(2-n-morpholinoethyl methacrylate), poly(1-vinylimidazole), poly(2-vinylpyridine), and (4-vinylpyridine).

11. A method of preparing pH independent drug release system wherein the method comprises applying a polymeric composite coating of claim 1 onto drug-loaded beads.

12. The method of claim 11, wherein the drug release retardant polymer matrix comprises any one or a combination of cellulose derivatives, (alkyl) acrylate polymers and derivatives, polyvinyls and copolymers.

13. The method of claim 11, wherein the pH-responsive nanoparticulate pore formers comprise a first polymer comprising a polysaccharide; a crosslinked second polymer comprising an ionizable polymer; and a polysorbate comprising a (C9-C31)R—C(O)O— group covalently bound to the second polymer by a C—C bond between the carbon backbone of the second polymer and the R group.

14. The method of claim 11, wherein the pH-responsive nanoparticulate pore formers function to modulate the permeability of the overall composite coating in response to changes in pH throughout the gastrointestinal tract.

15. The method of claim 11, wherein the drug is weakly basic or acidic.

16. A method of preparing alcohol resistant drug release system, said method comprising applying a polymeric composite coating of claim 1 onto drug-loaded beads.

17. The method of claim 16, wherein the drug release retardant polymer matrix comprises any one or a combination of cellulose derivatives, (alkyl) acrylate polymers and derivatives, polyvinyls and copolymers.

18. The method of claim 16, wherein the pH-responsive nanoparticulate pore formers function as alcohol-resistant component to the overall composite coating to resist increased solubility and permeability in presence of alcohol at 40% ethanol concentration in aqueous media.

19. The method of claim 16, wherein the pH-responsive nanoparticulate pore formers comprise a first polymer comprising a polysaccharide; a crosslinked second polymer comprising an ionizable polymer; and a polysorbate comprising a (C9-C31)R—C(O)O— group covalently bound to the second polymer by a C—C bond between the carbon backbone of the second polymer and the R group.

20. The method of claim 17, wherein the pH-responsive nanoparticulate pore formers comprise a first polymer comprising a polysaccharide; a crosslinked second polymer comprising an ionizable polymer; and a polysorbate comprising a (C9-C31)R—C(O)O— group covalently bound to the second polymer by a C—C bond between the carbon backbone of the second polymer and the R group.
